(19) United States
(12) Patent Application Publication
(10) Pub. No.: US 2013/0288909 A1
(43) Pub. Date: Oct. 31, 2013

(54) miRNA DETECTION OF PANCREATIC CANCER

(71) Applicants: Subrata Sen, Houston, TX (US); Jin Wang, Houston, TX (US)

(72) Inventors: Subrata Sen, Houston, TX (US); Jin Wang, Houston, TX (US)

(73) Assignee: BOARD OF REGENTS THE UNIVERSITY OF TEXAS SYSTEM, AUSTIN, TX (US)

(21) Appl. No.: 13/819,898

(22) PCT Filed: Aug. 31, 2011
(86) PCT No.: PCT/US11/49958

§ 371 Date: Jul. 15, 2013

Related U.S. Application Data
(60) Provisional application No. 61/378,712, filed on Aug. 31, 2010.

(51) Int. Cl. C12Q 1/68

(52) U.S. Cl. CPC C12Q 1/6827
USPC 506/3; 435/6.11; 506/16

(57) ABSTRACT

Methods for the diagnosis and/or monitoring of pancreatic cancer by detection of micro-RNAs (miRNA) are provided. In certain embodiments, detection of miR-21, miR-210, miR-155, and/or miR-196a in the plasma, blood, or pancreatic juice of a subject, such as a human patient, may be used to detect, diagnose, or monitor a pancreatic cancer.

This application claims priority to U.S. Application No. 61/378,712 filed on Aug. 31, 2010, the entire disclosure of which is specifically incorporated herein by reference in its entirety without disclaimer.

This invention was made with government support under UO1CA111302 awarded by the National Cancer Institute and CA16672 awarded by The National Cancer Institute Cancer Center. The government has certain rights in the invention.

### BACKGROUND OF THE INVENTION

1. Field of the Invention

The present invention relates generally to the fields of molecular biology and medicine. More particularly, it concerns methods for the diagnosis of cancer by detection of specific miRNA.

2. Description of Related Art

Biomarkers for the early diagnosis or detection of pancreatic cancer have been largely unavailable in the past. Pancreatic cancer is the fourth most common cause of cancer related deaths in the United States, and pancreatic cancer has an average 5-year survival rate of <5% (Maitra and Hruban, 2008). Most patients with this neoplasm remain asymptomatic until they present with locally advanced or distally metastatic and surgically inoperable disease at the time of diagnosis. A blood-based biomarker assay for malignancies such as pancreatic cancer could yield significant clinical benefit, since early detection of this malignancy at a surgically resectable stage is believed to offer the best curative option for the patients (Goggins, 2007). Sensitive and specific cancer biomarkers could help with early detection, diagnosis, and prevention strategies. Unfortunately, blood based biomarker assays have not been available for this purpose.

The majority of pancreatic cancers arise from the epithelial lining of the exocrine pancreatic ducts as pancreatic ductal adenocarcinoma (PDAC) through a multi-step progression process involving non-invasive precursor lesions (Hruban et al., 2007). The precursor lesions consist of microscopic pancreatic intraepithelial neoplasia (PanIN) within the duct (Hruban et al., 2004) and macroscopic intra-ductal papillary mucinous neoplasms (IPMN) as cystic lesions connected with the main pancreatic duct or one of its branches (Adsay et al., 2005). In both PanIN and IPMN, the epithelium lining demonstrates varying degrees of histologic atypia ranging from adenoma to carcinoma in situ (Hruban et al., 2004). Interestingly, some of the seminal genetic alterations detected in invasive pancreatic carcinomas, such as mutations in KRAS2, DPC4/SMAD4 and TP53 are observed in both PanIN (Lüttges et al., 1999; Wilentz et al., 2000; DiGiuseppe et al., 1994) and IPMN (Hruban et al., 2007; Maitra et al., 2005) lending support to their roles as bona fide precursor lesions.

Limitations in diagnostic methods and the lack of early-stage disease symptoms are considered to be the major cause of the high mortality rate associated with pancreatic cancer. Despite identification of such common genetic mutation signatures shared between non-invasive and invasive pancreatic lesions, their utility in discriminating between benign and malignant pancreatic disease remain unresolved primarily because minimally invasive diagnostic methods for screening of candidate biomarkers are still not available. It has been reported that even imaging techniques like computed tomography (CT) and magnetic resonance imaging (MRI) sometimes fail to differentiate between benign and malignant lesions with up to 6% of the cases suspected as malignant with these methods later found to be benign at surgery with subsequent post surgical complications developing among a significant number of these patients (Van Gulik et al., 1997). Endoscopic ultrasound guided fine needle aspiration (EUS-FNA) has emerged as the preferred procedure for preoperative diagnosis and staging of pancreatic cancer (Vilmann and Saftoiu, 2006; Chen et al., 2007; Eloubeidi et al., 2003; Jhala et al., 2006; Jhala et al., 2004). However, the invasive nature of the technique makes it unlikely that EUS-FNA could be routinely used for early detection or screening of pancreatic carcinomas. Clearly, there exists a need for the development of a minimally invasive biomarker assay that can be performed with plasma, serum or an accessible biological fluid for pancreatic cancer screening.

### SUMMARY OF THE INVENTION

The present invention overcomes limitations in the prior art by providing, in certain aspects, plasma, serum, and pancreatic juice based biomarker assays for pancreatic cancer screening that involve analysis of specific micro-RNA (miRNA). The present invention is based, in part, on the discovery that elevated levels of miR-21, miR-210, miR-155, and/or miR-196a in a blood, plasma, or pancreatic juice sample from a human subject can indicate the presence or an increased risk of a pancreatic cancer. Aspects of the present invention relate to the diagnosing or monitoring the progression of a cancer such as a pancreatic cancer, e.g., comprising an exocrine tumor or an endocrine tumor. In various aspects, specific and sensitive blood-based or pancreatic juice-based miRNA biomarker assays provided herein may be used to provide significant clinical benefit for the diagnosis and treatment of pancreatic cancer.

An aspect of the present invention relates to a method of detecting for the presence or an increased risk of a pancreatic cancer in a subject, comprising obtaining a biological sample that comprises miRNA sequences of the subject; and measuring the level of miR-21, miR-210, miR-155, and/or miR-196a in the biological sample, wherein an increased level of miR-21, miR-210, miR-155, and/or miR-196a relative to a normal control indicates an increased risk of or the presence of a pancreatic cancer in the subject. The biological sample may comprise or be selected from the group consisting of a blood sample, a plasma sample (e.g., a plasma sample containing EDTA), a serum sample, or a pancreatic juice sample. In some embodiments, the method comprises measuring the level of one, two, three or all of miR-21, miR-210, miR-155, and/or miR-196a in the biological sample. In some embodiments, if the biological sample comprises pancreatic juice, then said measuring comprises measuring miR-210 or miR-196a in the biological sample. In some embodiments, the pancreatic cancer is not a non-invasive intraductal papillary mucinous neoplasm (IPMN) of the pancreas. The pancreatic cancer may be metastatic or non-metastatic. The pancreatic juice sample may be a secretin stimulated exocrine pancreatic secretions (SSEPS) sample. Said measuring may comprise measuring the level of said miR-21, miR-210, miR-155, or miR-196a using RT-PCR, a biochip, quantitative PCR, serial analysis of gene expression (SAGE), or a microarray. Said measuring may comprise measuring the level of at least two, at least three, or all of said miR-21, miR-210, miR-155, or miR-196a. In some embodiments, increased levels of miR-21 and/or miR-210 in a pancreatic juice sample indicates the presence of or an increased risk of the pancreatic cancer subject. The subject may be a human. The human may have pancreatic cancer, pancreatitis, or chronic pancreatitis. In some embodiments, the method further comprises a method of discriminating between a pancreatic cancer and pancreatitis in the subject, wherein an increase in the level of miR-196a relative to a normal control indicates that the subject indicates an increased risk of or the presence of a pancreatic cancer in the subject; wherein no or substantially no increase in the level of miR-196a, and an increase in at least one of miR-21, miR-210, or miR-155 indicates that the subject has pancreatitis or chronic pancreatitis.

Another aspect of the present invention relates to a method for monitoring the progression of a pancreatic cancer in a subject comprising: obtaining a first biological sample from the subject; subsequently obtaining a second biological sample from the subject, and measuring miR-21, miR-210, miR-155, and/or miR-196a levels in said first and second biological samples; wherein an increase in the expression of miR-21, miR-210, miR-155, and/or miR-196a in the second sample relative to the first sample indicates an adverse disease progression of the pancreatic cancer in the patient, and wherein a decrease in the expression of miR-21, miR-210, miR-155, and/or miR-196a in the second sample relative to the first sample indicates disease regression of the pancreatic cancer in the patient. The biological sample may comprise or be selected from the group consisting of a blood, serum, or pancreatic juice sample. The subject may be a human patient. In various embodiments, one, two, three, or all of miR-21, miR-210, miR-155, and/or miR-196a are measured in the first and second samples. In some embodiments, said measuring comprises measuring miR-196a, miR-210, or miR-155 in the biological sample. In some embodiments, the pancreatic juice sample comprises or consists of a SSEPS sample. Said measuring may comprise using RT-PCR, a biochip, quantitative PCR, serial analysis of gene expression (SAGE), or a microarray. The method may further comprise measuring at least one of miR-21, miR-210, miR-155 in said first and second biological samples. Said measuring may comprise measuring expression of said miR-21, miR-210, miR-155, and miR-196a in said first and second biological samples. The patient may be administered an anti-cancer therapy, such as, e.g., a surgery, a chemotherapy, a radiation therapy, a gene therapy, a protein therapy, or an immunotherapy.

As used herein, “obtaining a biological sample” refers to receiving a biological sample, e.g., either directly or indirectly. For example, in some embodiments, the biological sample, such as a blood sample or a pancreatic juice sample, is directly obtained from a subject by a laboratory technician, doctor, nurse, or medical professional. In other embodiments, the biological sample may be drawn or taken by a third party and then transferred, e.g., to a separate entity or location for analysis (e.g., detecting or measuring one or more miRNA). In these embodiments, said obtaining refers to receiving the sample, e.g., from the mail, courier, or post office, etc. In some further aspects, the method may further comprise reporting the determination to the subject, a health care payer, an attending clinician, a pharmacist, a pharmacy benefits manager, or any person that the determination may be of interest.

Yet another aspect of the present invention relates to a kit comprising a sealed container comprising primers or probes designed to detect specific for transcription or reverse transcription of at least two, at least three, or all of miR-21, miR-210, miR-155, and/or miR-196a. The kit may further comprise one or more reagents for RT-PCR or reverse transcription RT-PCR.

Another aspect of the present invention relates to a method for discriminating or distinguishing between a pancreatic cancer and pancreatitis in a human subject comprising: obtaining a biological sample from the subject; measuring miR-196a levels in the biological sample, and measuring at least one of miR-21, miR-210, miR-155 in the biological sample; wherein an increase in the expression of miR-196a in the sample relative to a normal control indicates the presence of or an increased risk of a pancreatic cancer in the subject; and wherein the lack of an increase in the level of miR-196a relative to a normal control, and an increase in one or more of miR-21, miR-210, or miR-155 relative to a normal control, indicates that the subject has the presence of or an increased of pancreatitis; wherein the biological sample comprises a blood sample, a serum sample, or a pancreatic juice sample. The measuring may comprise using RT-PCR, a biochip, quantitative PCR, serial analysis of gene expression (SAGE), or a microarray. The method may comprise measuring at least two, at least three, or all of miR-21, miR-210, and miR-155 in the biological sample. The subject may have a pancreatic cancer, such as an exocrine tumor or an endocrine tumor. In some embodiments, the subject has pancreatitis, such as chronic pancreatitis or acute pancreatitis. The subject may be administered an anti-cancer therapy, such as a surgery, a chemotherapy, a radiation therapy, a gene therapy, a protein therapy, or an immunotherapy.

Another aspect of the present invention relates to a biochip comprising an isolated nucleic acid comprising at least two, at least three of miR-21, miR-210, miR-155, or miR-196a, or a complement thereof. The bochip may comprise each of miR-21, miR-210, miR-155, or miR-196a, or its complement.

Although, in certain embodiments, human subjects may be tested for the presence or an increased risk of a pancreatic cancer, it is anticipated that the methods may be used to test a non-human mammal, such as a dog, cat, horse, sheep, rat, mouse, or non-human primate, e.g., utilizing circulating miRNAs.

The use of the word “a” or “an” when used in conjunction with the term “comprising” in the claims and/or the specification may mean “one,” but it is also consistent with the meaning of “one or more,” “at least one,” and “one or more than one.”

It is contemplated that any embodiment discussed in this specification can be implemented with respect to any method or composition of the invention, and vice versa. Furthermore, compositions of the invention can be used to achieve methods of the invention.

Throughout this application, the term “about” is used to indicate that a value includes the inherent variation of error for the device, the method being employed to determine the value, or the variation that exists among the study subjects.

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-D: The relative fold change of four miRNAs in the plasma of pancreatic adenocarcinoma patients and normal healthy controls. The horizontal line of pluses (+) represent the mean fold change for each miRNA. miR-21 (FIG. 1A), miR-210 (FIG. 1B), miR-155 (FIG. 1C), and miR-196a (FIG. 1D) are shown.

FIGS. 2A-E: Receiver operating characteristic (ROC) curves of different sample sets analyzed for the plasma levels of the four individual miRNAs (FIGS. 2A-D) and an identical sample set analyzed for the four miRNA individually and in combination as a composite panel (FIG. 2E).

FIGS. 3A-H: Receiver operating characteristic (ROC) curves of different sample sets analyzed for the PDAC pancreatic juice levels of the four individual miRNAs (FIGS. 3A-D), an identical sample set analyzed for the four miRNAs as a composite panel (FIG. 3E), an identical sample set analyzed for CA19-9 (FIG. 3F), four miRNA panel and CA19-9 combination (FIG. 3G) and miRNA panel, CA19-9, alcohol and smoking combination (FIG. 3H)

FIGS. 4A-H: Receiver operating characteristic (ROC) curves of different sample sets analyzed for the pancreatitis (CP) pancreatic juice levels of the four individual miRNAs (FIGS. 4A-D), an identical sample set analyzed for the four miRNAs as a composite panel (FIG. 4E), an identical sample set analyzed for CA19-9 (FIG. 4F), four miRNA panel and CA19-9 combination (FIG. 4G) and miRNA panel, CA19-9, alcohol and smoking combination (FIG. 4H)

### DESCRIPTION OF ILLUSTRATIVE EMBODIMENTS

The present invention is based, in part, on the discovery that increased expression of miR-21, miR-210, miR-155, and/or miR-196a in a biological sample, such as a blood, blood plasma, blood serum, or pancreatic juice sample from a patient can indicate the presence, increased risk, or progression of a cancer, such as a pancreatic cancer. Methods and kits are provided for the detection of miR-21, miR-210, miR-155, and/or miR-196a for diagnosing or monitoring the progression of a cancer. Pancreatic cancers that may be detected or monitored include exocrine tumors and endocrine tumors (also referred to as neuroendocrine tumors or islet cell tumors).

### I. DEFINITIONS

“Attached” or “immobilized” as used herein to refer to a nucleic acid probe and a solid support may mean that the binding between the probe and the solid support is sufficient to be stable under conditions of binding, washing, analysis, and removal. The binding may be covalent or non-covalent. Covalent bonds may be formed directly between the probe and the solid support or may be formed by a cross linker or by inclusion of a specific reactive group on either the solid support or the probe or both molecules. Non-covalent binding may be one or more of electrostatic, hydrophilic, and hydrophobic interactions. Included in non-covalent binding is the covalent attachment of a molecule, such as streptavidin, to the support and the non-covalent binding of a biotinylated probe to the streptavidin. Immobilization may also involve a combination of covalent and non-covalent interactions.

“Complement” or “complementary” as used herein to refer to a nucleic acid may mean Watson-Crick (e.g., A-T/U and C-G) or Hoogsteen base pairing between nucleotides or nucleotide analogs of nucleic acid molecules.

“Differential expression” may mean qualitative or quantitative differences in the temporal and/or cellular gene expression patterns within and among cells and tissue. Thus, a differentially expressed gene may qualitatively have its expression altered, including an activation or inactivation, in, e.g., normal versus disease tissue. Genes may be turned on or turned off in a particular state, relative to another state thus permitting comparison of two or more states. A qualitatively regulated gene may exhibit an expression pattern within a state or cell type which may be detectable by standard techniques. Some genes may be expressed in one state or cell type, but not in both. Alternatively, the difference in expression may be quantitative, e.g., in that expression is modulated, either up-regulated, resulting in an increased amount of transcript, or down-regulated, resulting in a decreased amount of transcript. The degree to which expression differs need only be large enough to quantify via standard characterization techniques such as expression arrays, quantitative reverse transcriptase PCR, northern analysis, and RNase protection.

“Gene” used herein may be a natural (e.g., genomic) or synthetic gene comprising transcriptional and/or translational regulatory sequences and/or a coding region and/or non-translated sequences (e.g., introns, 5′- and 3′-untranslated sequences). The coding region of a gene may be a nucleotide sequence coding for an amino acid sequence or a functional RNA, such as tRNA, rRNA, catalytic RNA, siRNA, miRNA or antisense RNA. A gene may also be an mRNA or cDNA corresponding to the coding regions (e.g., exons and miRNA) optionally comprising 5′- or 3′-untranslated sequences linked thereto. A gene may also be an amplified nucleic acid molecule produced in vitro comprising all or a part of the coding region and/or 5′- or 3′-untranslated sequences linked thereto.

“Identical” or “identity” as used herein in the context of two or more nucleic acids or polypeptide sequences, may mean that the sequences have a specified percentage of residues that are the same over a specified region. The percentage may be calculated by optimally aligning the two sequences, comparing the two sequences over the specified region, determining the number of positions at which the identical residue occurs in both sequences to yield the number of matched positions, dividing the number of matched positions by the total number of positions in the specified region, and multiplying the result by 100 to yield the percentage of sequence identity. In cases where the two sequences are of different lengths or the alignment produces one or more staggered ends and the specified region of comparison includes only a single sequence, the residues of single sequence are included in the denominator but not the numerator of the calculation. When comparing DNA and RNA, thymine (T) and uracil (U) may be considered equivalent. Identity may be performed manually or by using a computer sequence algorithm such as BLAST or BLAST 2.0.

“Label” as used herein may mean a composition detectable by spectroscopic, photochemical, biochemical, immunochemical, chemical, or other physical means. For example, useful labels include ³²P, fluorescent dyes, electron-dense reagents, enzymes (e.g., as commonly used in an ELISA), biotin, digoxigenin, or haptens and other entities which can be made detectable. A label may be incorporated into nucleic acids and proteins at any position.

“Nucleic acid” or “oligonucleotide” or “polynucleotide” used herein may mean at least two nucleotides covalently linked together. The depiction of a single strand also defines the sequence of the complementary strand. Thus, a nucleic acid also encompasses the complementary strand of a depicted single strand. Many variants of a nucleic acid may be used for the same purpose as a given nucleic acid. Thus, a nucleic acid also encompasses substantially identical nucleic acids and complements thereof. A single strand provides a probe that may hybridize to a target sequence under stringent hybridization conditions. Thus, a nucleic acid also encompasses a probe that hybridizes under stringent hybridization conditions. Nucleic acids may be single stranded or double stranded, or may contain portions of both double stranded and single stranded sequence. The nucleic acid may be DNA, both genomic and cDNA, RNA, or a hybrid, where the nucleic acid may contain combinations of deoxyribo- and ribo-nucleotides, and combinations of bases including uracil, adenine, thymine, cytosine, guanine, inosine, xanthine hypoxanthine, isocytosine and isoguanine. Nucleic acids may be obtained by chemical synthesis methods or by recombinant methods.

A nucleic acid will generally contain phosphodiester bonds, although nucleic acid analogs may be included that may have at least one different linkage, e.g., phosphoramidate, phosphorothioate, phosphorodithioate, or O-methylphosphoroamidite linkages and peptide nucleic acid backbones and linkages. Other analog nucleic acids include those with positive backbones; non-ionic backbones, and non-ribose backbones, including those described in U.S. Pat. Nos. 5,235,033 and 5,034,506, which are incorporated by reference. Nucleic acids containing one or more non-naturally occurring or modified nucleotides are also included within one definition of nucleic acids. The modified nucleotide analog may be located for example at the 5′-end and/or the 3′-end of the nucleic acid molecule. Representative examples of nucleotide analogs may be selected from sugar- or backbone-modified ribonucleotides. It should be noted, however, that also nucleobase-modified ribonucleotides, i.e. ribonucleotides, containing a non-naturally occurring nucleobase instead of a naturally occurring nucleobase such as uridines or cytidines modified at the 5-position, e.g. 5-(2-amino) propyl uridine, 5-bromo uridine; adenosines and guanosines modified at the 8-position, e.g. 8-bromo guanosine; deaza nucleotides, e.g. 7-deaza-adenosine; O- and N-alkylated nucleotides, e.g. N6-methyl adenosine are suitable. The 2′-OH-group may be replaced by a group selected from H, OR, R, halo, SH, SR, NH₂, NHR, NR₂ or CN, wherein R is C₁-C₆ alkyl, alkenyl or alkynyl and halo is F, Cl, Br or I. Modified nucleotides also include nucleotides conjugated with cholesterol through, e.g., a hydroxyprolinol linkage as described in Krutzfeldt et al. (2005); Soutschek et al. (2004); and U.S. Patent Publication No. 20050107325, which are incorporated herein by reference. Modified nucleotides and nucleic acids may also include locked nucleic acids (LNA), as described in U.S. Patent Publication No. 2002/0115080, U.S. Pat. No. 6,268,490, and U.S. Pat. No. 6,770,748, which are incorporated herein by reference. LNA nucleotides include a modified extra methylene “bridge” connecting the 2′ oxygen and 4′ carbon of the ribose ring. The bridge “locks” the ribose in the 3′-endo (North) conformation, which is often found in the A-form of DNA or RNA. LNA nucleotides can be mixed with DNA or RNA bases in the oligonucleotide whenever desired. Such oligomers are commercially available from companies including Exiqon (Vedbaek, Denmark). Additional modified nucleotides and nucleic acids are described in U.S. Patent Publication No. 20050182005, which is incorporated herein by reference. Modifications of the ribose-phosphate backbone may be done for a variety of reasons, e.g., to increase the stability and half-life of such molecules in physiological environments, to enhance diffusion across cell membranes, or as probes on a biochip. Mixtures of naturally occurring nucleic acids and analogs may be made; alternatively, mixtures of different nucleic acid analogs, and mixtures of naturally occurring nucleic acids and analogs may be made.

“Stringent hybridization conditions” used herein may mean conditions under which a first nucleic acid sequence will hybridize to a second nucleic acid sequence, such as in a complex mixture of nucleic acids. Stringent conditions are sequence-dependent and will be different in different circumstances. Stringent conditions may be selected to be about 5-10° C. lower than the thermal melting point (Tₘ) for the specific sequence at a defined ionic strength pH. The Tₘ may be the temperature (under defined ionic strength, pH, and nucleic concentration) at which 50% of the probes complementary to the target hybridize to the target sequence at equilibrium (as the target sequences are present in excess, at Tₘ, 50% of the probes are occupied at equilibrium). Stringent conditions may be those in which the salt concentration is less than about 1.0 M sodium ion, such as about 0.01-1.0 M sodium ion concentration (or other salts) at pH 7.0 to 8.3 and the temperature is at least about 30° C. for short probes (e.g., about 10-50 nucleotides) and at least about 60° C. for long probes (e.g., greater than about 50 nucleotides). Stringent conditions may also be achieved with the addition of destabilizing agents such as formamide. For selective or specific hybridization, a positive signal may be at least 2 to 10 times background hybridization. Exemplary stringent hybridization conditions include the following: 50% formamide, 5×SSC, and 1% SDS, incubating at 42° C., or, 5×SSC, 1% SDS, incubating at 65° C., with wash in 0.2×SSC, and 0.1% SDS at 65° C.

“miRNA sequence” as used herein refers to a nucleic acid comprising a miRNA or a nucleic acid (e.g., a cDNA or RNA) which is complementary to a miRNA. For example, in certain embodiments, miRNA may be obtained and/or measured from a biological sample. Alternately, a miRNA may be reverse transcribed to a cDNA, and the cDNA may be measured (e.g., using real time PCR) to assess levels of miRNA in the biological sample.

“Substantially complementary” used herein may mean that a first sequence is at least 60%, 65%, 70%, 75%, 80%, 85%, 90%, or 95% identical to the complement of a second sequence over a region of 8, 9, 10, 11, 12, 13, 14, 15, 16, 17, 18, 19, 20, 21, 22, 23, 24, or more nucleotides, or that the two sequences hybridize under stringent hybridization conditions.

### II. MIRNA

MicroRNAs (miRNAs) are 18-24 nucleotide long evolutionarily conserved RNA molecules (Chang and Mendell, 2007) that regulate the stability and translational efficiency of target mRNAs by complementary base pairing with specific 3′UTR sequences. Such physiologic regulation of transcriptome function by miRNAs plays a significant role in the maintenance of cellular homeostasis and development.

As described in the below examples, plasma profiles of a set of miRNAs were found to discriminate pancreatic cancer patients from normal healthy individuals. These results demonstrate that the levels of known pancreatic cancer associated miRNAs may be elevated in the plasma and/or pancreatic juice of pancreatic carcinoma patients and that combined analyses of these circulating miRNAs may be used to differentiate cancer patients from healthy individuals. In some embodiments, the increased levels miR-21, miR-210, or miR-155 may be used to detect or monitor pancreatitis in a subject.

In various embodiments, increased levels of miR-21, miR-210, miR-155, and/or miR-196a in a blood or plasma sample from a human subject can indicate the presence or an increased risk of a pancreatic cancer. miR-21 (MIMAT0000076, UAGCUUAUCAGACUGAUGUUGA (SEQ ID NO:1)), miR-210 (MIMAT0000267, CUGUGCGUGUGACAGCGGCUGA (SEQ ID NO:2)), miR-155 (MIMAT0000646, UUAAUGCUAAUCGUGAUAGGGGU (SEQ ID NO:3)), and miR-196a (MIMAT0000226, UAGGUAGUUUCAUGUUGUUGGG (SEQ ID NO:4)) are miRNA which are known in the art.

It is anticipated that one or more of the following miRNAs may be elevated in the blood or plasma of a subject and may correlate with the presence of or an increased risk of pancreatic cancer: hsa-miR-200b (MIMAT0000318, UAAUACUGCCUGGUAAUGAUGA (SEQ ID NO:5)), hsa-miR-190 (MIMAT0000458, UGAUAUGUUUGAUAUAUUAGGU (SEQ ID NO:6)), hsa-miR-186 (MIMAT0000456, CAAAGAAUUCUCCUUUUGGGCU (SEQ ID NO:7)), hsa-miR-221 (MIMAT0000278, AGCUACAUUGUCUGCUGGGUUUC (SEQ ID NO:8)), hsa-miR-222 (MIMAT0000279, AGCUACAUCUGGCUACUGGGU (SEQ ID NO:9)), hsa-miR-15b (MIMAT0000417, UAGCAGCACAUCAUGGUUUACA (SEQ ID NO:10)), hsa-miR-95 (MIMAT0000094, UUCAACGGGUAUUUAUUGAGCA (SEQ ID NO:11)), hsa-miR-223 (MIMAT0000280, UGUCAGUUUGUCAAAUACCCCA (SEQ ID NO:12)), hsa-miR-203 (MIMAT0000264, GUGAAAUGUUUAGGACCACUAG (SEQ ID NO:13)).

One, two, three or all of miR-21, miR-210, miR-155, and/or miR-196a may be measured in a biological sample from a subject, and used to diagnose the presence or an increased risk of a cancer, wherein increased expression of one, two, three or all of miR-21, miR-210, miR-155, and/or miR-196a indicates the presence of or an increased risk of a cancer. For example, as shown in the below examples, elevated plasma levels of miR-155 and miR-210 had a statistically significant association with the presence of a cancer in a patient. In certain embodiments, increased levels of miR-155 and miR-210 can indicate the presence or an increased risk of a cancer. The cancer may be a pancreatic cancer, such as a pancreatic ductal adenocarcinoma, a tubular adenoma, or Intrapapillary mucinous neoplasm (IPMN). In other embodiments, increased expression of one, two, three or all of miR-21, miR-210, miR-155, and/or miR-196a in a biological sample, such as a blood or serum sample, from a patient may indicate the presence of a non-pancreatic cancer in a patient, such as breast cancer, esophageal cancer, gastric cancer, glioblastoma, head and neck cancer, lung cancer, lymphoma, osteosarcoma, ovarian cancer or prostate cancer. For example, increased circulating levels of miR-155 and miR-210 may indicate the presence or increased risk of breast cancer, head and neck cancer, lung cancer, lymphoma or ovarian cancer.

### III. METHODS FOR ANALYZING EXPRESSION OF MIRNA AND GENE EXPRESSION

Some embodiments of the methods of the present invention involve analysis of miRNA expression or gene expression, such as miR-21, miR-210, miR-155, and/or miR-196a. Methods for analyzing gene expression or expression of miRNA include, but are not limited to, methods based on hybridization analysis of polynucleotides, sequencing of polynucleotides, and analysis of protein expression such as proteomics-based methods. Commonly used methods for the quantification of mRNA expression in a sample include northern blotting and in situ hybridization (Parker and Barnes, 1999), RNAse protection assays (Hod, 1992), and PCR-based methods, such as reverse transcription polymerase chain reaction (RT-PCR) (Weis et al., 1992). In some embodiments, antibodies may be employed that can recognize specific duplexes, including DNA duplexes, RNA duplexes, and DNA-RNA hybrid duplexes or DNA-protein duplexes. Representative methods for sequencing-based gene expression analysis include Serial Analysis of Gene Expression (SAGE), and gene expression analysis by massively parallel signature sequencing (MPSS).

Increased levels of miR-21, miR-210, miR-155, and/or miR-196a in a biological sample, such as a blood, serum, or pancreatic juice sample, can indicate the presence of, an increased risk of, or progression of a cancer, such as a pancreatic cancer. In various embodiments an increase of about 2.5 fold, about 3 fold, about 3.5 fold, about 4 fold, about 4.5 fold, or about 5 fold or more in the levels of miR-21, miR-210, miR-155, and/or miR-196a in the biological sample can indicate the presence of, an increased risk of, or progression of a cancer, such as a pancreatic cancer. In certain embodiments, a 5 fold increase in the expression of miR-21, miR-210, miR-155, and/or miR-196a in the biological sample may be used as the cutoff range to determine whether or not a subject has, or is at increased risk of, a pancreatic cancer. Measuring levels of miR-21, miR-210, miR-155, or miR-196a may be performed directly or indirectly via methods including, e.g., direct measurement of the miRNA or by reverse transcription of the mRNA into cDNA and then measurement of the cDNA (e.g., as performed in reverse transcription RT-PCR).

In certain preferred embodiments, levels of circulating miR-21, miR-210, miR-155, and/or miR-196a are measured in a blood, serum, or plasma sample from a patient. Nonetheless, it is anticipated that other biological samples may be used for this purpose. In various embodiments, the biological sample may comprise a tissue biopsy, or serum, or plasma, or pancreatic juice or endoscopic ultrasound guided fine needle aspirate (EUS-FNA) may be used. In some embodiments, the biological sample is not a tissue biopsy. In some embodiments, a blood or plasma sample may be collected in or placed into a vessel or container comprising EDTA (ethylenediaminetetraacetic acid). In various embodiments, or EDTA may be added to a blood or plasma sample.

A. Blood and Pancreatic Juice Based Methods

Major ongoing research efforts in the field of cancer detection and diagnosis concentrate on the development of biomarker assays that could be performed on blood samples or other body fluids involving non-invasive or minimally invasive techniques. Assays are preferably capable of differentiating patients with precursor and advanced malignant lesions at different stages of malignancy from normal healthy individuals. Blood plasma and serum may, in certain embodiments, be preferred choices for developing such detection, diagnostic and prognostic markers. Several studies have attempted to utilize proteomic profiling of plasma and serum samples to identify peptide biomarkers reflective of physiologic or pathologic state of malignancy in human cancer patients as well as in genetically engineered mouse models of cancer (Faca et al., 2008). However, with the challenge of tumor associated proteins possibly constituting only a minor fraction within the vastly abundant dynamic range of plasma proteins, proteomic based strategies of cancer biomarker identification have had limited success to date (Hanash et al., 2008). miRNAs may be present in blood plasma and serum, and certain miRNAs have correlated with prostate cancer (Mitchell et al., 2008). miRNA expression profiles in cancerous tissues have been associated with various human cancer tissues including breast, lung, esophagus, prostate and pancreas. Differential expression of miRNAs may correlate with important histopathologic features like tumor stage, proliferation capacity and vascular invasion (Lynam-Lennon et al., 2009).

Pancreatic juice samples, or a tissue sample comprising a pancreatic tissue secretion, may be used to detect the presence of or an increased risk of a pancreatic cancer in a patient. Pancreatic juice may be obtained as previously described, e.g., in Pungpapong et al. (2007), Matsuo et al. (2009), and Noh et al. (2006). As observed in the below examples, alterations in one or more miRNA were observed to be associated with the presence of a pancreatic cancer. Pancreatic juice includes and, in some embodiments, refers to secretin-stimulated exocrine pancreatic secretions (SSEPS). SSEPS is generally less invasive as compared to ERCP or pancreatic duct cannulation. In some embodiments, in addition to measuring or detecting miRNA in SSEPS or pancreatic juice from a patient, the pancreatic juice or SSEPS may also be tested to measure the presence or amount of one or more proteins, glycoproteins, lipids, and/or glycolipids.

In various embodiments, SSEPS may be collected from a patient via the following method. Synthetic human secretin [ChiRhoClin] may be administered to a subject (e.g., about 0.25 μg/Kg or about 16 μg total) via i.v. Next, SSEPS may be collected about 1 min later with 7Fr catheter from ampulla for about 10 min. Collected SSEPS (in 20 ml tube) may be divided into aliquots of 2 ml with 1 tablet of protease inhibitor cocktail (Roche). Collected SSEPS may then be snap frozen. In some embodiments, the pancreatic juice may contain a plurality of pancreatic cells or pancreatic tissue. In other embodiments, the pancreatic juice is free or essentially free of pancreatic cells or pancreatic tissue.

As shown in the below examples, plasma miRNA analyses can be used to differentiate pancreatic adenocarcinoma patients from healthy controls. These results support the idea that miRNA profiling in blood plasma or pancreatic juice (e.g., SSEPS) may be used as a minimally invasive biomarker assay for pancreatic cancer. Developing a specific and sensitive blood-based or pancreatic juice-based miRNA biomarker assay may be particularly useful for the diagnosis and treatment of pancreatic cancer since current limitations in diagnostic methods and lack of early stage disease symptoms are considered the major cause of high mortality rate among these patients.

Four miRNAs: miR-21, miR-210, miR-155 and miR-196a were chosen for evaluation in the below examples. This biased approach for the analysis of only a few selected miRNAs supports their utility in blood plasma based biomarker assays for pancreatic cancer.

Without wishing to be bound by any theory, detection of increased levels of miR-21, miR-210, miR-155 and miR-196a circulating in the blood may provide insight into the biological characteristics of a pancreatic cancer. In pancreatic cancer, varying expression profiles of a number of miRNAs distinguishing malignant lesions from normal pancreatic tissue and chronic pancreatitis has been observed (Bloomston et al., 2007; Szafranska et al., 2007; Roldo et al., 2006). Among the four miRNAs analyzed in the below examples, up-regulation of miR-21 in cancer cells may be associated with apoptosis inhibition and acquisition of invasive properties (Chan et al., 2005; Asangani et al., 2008), possibly mediated by its down-regulating effects on the expression of two target tumor suppressor genes PTEN (Yang et al., 2008) and PDCD4 (Frankel et al., 2008). Increased expression of miR-210, on the other hand, may be regulated in a hypoxia inducible factor-1 alpha dependent manner with possible downstream effects on DNA repair genes affecting genomic instability (Crosby et al., 2009). A functional role for miR-155 in pancreatic cancer has been implied based on the observations that it represses the function of the pro-apoptotic protein TP53INP1, which enhances tumorigenicity of pancreatic cancer cells in vivo (Gironella et al., 2007). Interestingly, miR-155 has been also identified as a biomarker of early pancreatic neoplasia consequent to the finding that it is over expressed in about 80% of precursor IPMN lesions (Habbe et al., 2009). Finally, over expression of miR-196a paralleling disease progression was reported to be a predictor of survival for pancreatic cancer patients (Bloomston et al., 2007; Szafranska et al., 2007). The results in the below examples demonstrate that combined analyses of these four miRNAs in plasma can discriminate pancreatic adenocarcinoma patients from normal healthy individuals with sensitivity and specificity.

In various embodiments, the following method may be used to test for miRNA in a biological sample such as a blood sample. A blood sample may be obtained from a patients in a container comprising an anticoagulant such as heparin. Total RNA containing small RNA may be isolated from 1.5 ml of heparin plasma using, e.g., Trizol™ LS reagent (Invitrogen Life Technologies, CA). The aqueous phase may then extracted twice with phenol/chloroform and added with 1.5 vol of ethanol before being applied directly to a mirVana miRNA column (Ambion, Inc, Austin, Tex.) according to the manufacturer's instructions. The bound RNA may be cleaned with the buffers provided by the manufacturer to remove impurities and eluted in a final volume of 100 μl. To remove heparin associated contaminants about 300 μl of 7.5 M LiCl may be added to the RNA solution, incubated overnight at about −20° C. and then centrifuged at about 12,000×g for about 30 min at about 4° C. The pellet may be washed two times by centrifugation with 70% ethanol. The RNA pellet may be dried for 10 min at room temperature and then dissolved in about 30 μl of DEPC-treated water for miRNA assay. The concentration of all RNA samples may be quantified, e.g., using NanoDrop 1000 (Nanodrop, Wilmington, USA). About 20 ng of RNA may be pretreated with 1 unit of DNase (Invitrogen, CA) and 2 units of Heparinase I (Sigma Chemical Company) for about 1 h at about 25° C. in about 1 μl of 10×DNase I Reaction Buffer (200 mM Tris-HCl pH 8.4, 20 mM MgCl2, 500 mM KCl) and about 0.38 μl of RNAse inhibitor to remove any contaminating DNA or heparin. After the enzyme digestions, about 0.5 μl of 25 mM EDTA solution may be added to the total reaction volume of 10 μl and incubated at about 65° C. for about 10 min. miRNA in the samples may then be measured, e.g., using real time RT-PCR.

B. PCR-Based Methods

Gene expression or miRNA expression can be analyzed using techniques that employ PCR. PCR is useful to amplify and detect transcripts from a sample. RT-PCR is a sensitive quantitative method that can be used to compare mRNA levels in different samples (e.g., endomyocardial biopsy samples) to examine gene expression signatures.

To perform RT-PCR, mRNA is isolated from a sample. For example, total RNA may be isolated from a biological sample. mRNA may also be extracted, for example, from frozen or archived paraffin-embedded and fixed tissue samples. Methods for mRNA extraction are known in the art. See, e.g., Ausubel et al. (1997). Methods for RNA extraction from paraffin embedded tissues are disclosed, for example, in Rupp and Locker, 1987, and De Andres et al., 1995. Purification kits for RNA isolation from commercial manufacturers, such as Qiagen, can be used. Other commercially available RNA isolation kits include MasterPure™. Complete DNA and RNA Purification Kit (EPICENTRE, Madison, Wis.), and, Paraffin Block RNA Isolation Kit (Ambion, Inc.). Total RNA from tissue samples can be also isolated using RNA Stat-60 (Tel-Test) or by cesium chloride density gradient centrifugation.

RNA may then be reverse transcribed into cDNA. The cDNA is amplified in a PCR reaction. A variety of reverse transcriptases are known in the art. For example, extracted RNA can be reverse-transcribed using a GeneAmp RNA PCR kit (Perkin Elmer, Calif., USA), following the manufacturer's instructions. The derived cDNA can then be used as a template in the subsequent PCR reaction.

For quantitative PCR, a third oligonucleotide, or probe, is used to detect nucleotide sequence located between the two PCR primers. The probe may be non-extendible by Taq DNA polymerase enzyme, and typically is labeled with a reporter fluorescent dye and a quencher fluorescent dye. Any laser-induced emission from the reporter dye is quenched by the quenching dye when the two dyes are located close together as they are on the probe. During the amplification reaction, the Taq DNA polymerase enzyme cleaves the probe in a template-dependent manner. The resultant probe fragments disassociate in solution, and signal from the released reporter dye is free from the quenching effect of the second fluorophore. One molecule of reporter dye is liberated for each new molecule synthesized, and detection of the unquenched reporter dye provides the basis for quantitative analysis.

RT-PCR can be performed using commercially available equipment, such as an ABI PRISM 7700™ Sequence Detection System (Perkin-Elmer-Applied Biosystems, Foster City, Calif., USA), or Lightcycler™ (Roche Molecular Biochemicals, Mannheim, Germany). Samples can be analyzed using a real-time quantitative PCR device such as the ABI PRISM 7700™ Sequence Detection System™.

A variation of the RT-PCR technique is real time quantitative PCR, which measures PCR product accumulation through a dual-labeled fluorigenic probe, such as a TaqMan™ probe. Real time PCR is compatible both with quantitative competitive PCR, where internal competitor for each target sequence is used for normalization, and with quantitative comparative PCR using a normalization gene contained within the sample, or a housekeeping gene for RT-PCR.

Gene expression may be examined using fixed, paraffin-embedded tissues as the RNA source or fresh tissue such as tissue obtained from a biopsy of pulmonary tissue. Examples of methods of examining expression in fixed, paraffin-embedded tissues, are described, for example, in Godfrey et al., 2000; and Specht et. al., 2001.

In certain embodiments, the following real-time RT-PCR protocol may be used to measure specific miRNA, such as miR-21, miR-210, miR-155, and/or miR-196a in a sample. Taqman™ MicroRNA Assays may be used to do expression profiling of the plasma miRNAs of interest. Reagents, primers, and probes may be obtained from Applied Biosystems (Applied Biosystems, Foster City, Calif.). About 10 ng of DNAse and Heparinase treated plasma RNA for each sample may be used for the individual assays in about 15 μl reactions containing RT mixture and Taqman™ primer mix. The mix may be incubated at about 16° C. for about 30 min, about 42° C. for about 60 min, and about 85° C. for about 5 min. miRNA expression levels may be quantified using the ABI prim 7900 HT Sequence detection system (Applied Biosystems). About 15 μl reverse transcription (RT) reaction may be diluted with about 30 μl of water and about 11.25 μl of the diluted RT product may be mixed with about 12.5 μl of 2×Taqman PCR mixture, about 1.25 μl Taqman primer and probe mixture in a final volume of about 25 μl. Real time PCR may be performed in triplicate, including no-template controls. Relative expression of the mature miRNAs may be calculated utilizing the comparative CT (2^{-ΔΔCT}) method (Schmittgen and Livak, 2008) with miRNA-16 as the endogenous control to normalize the data (Wong et al., 2008). The cycle threshold (CT) is defined as the number of cycles required for the FAM signal to cross the threshold in real time PCR. ΔCT may be calculated by subtracting the CT values of miR-16 from the CT values of the miRNA of interest. ΔΔCT may then be calculated by subtracting mean ΔCT of the control samples from ACT of tested samples. Fold change of miRNA may be calculated by the equation 2^{-ΔΔCT}.

Another approach for gene expression analysis employs competitive PCR design and automated, high-throughput matrix-assisted laser desorption ionization time-of-flight (MALDI-TOF) MS detection and quantification of oligonucleotides. This method is described by Ding and Cantor, 2003. See also the MassARRAY-based gene expression profiling method, developed by Sequenom, Inc. (San Diego, Calif.).

Additional PCR-based techniques for gene expression analysis include, e.g., differential display (Liang and Pardee, 1992); amplified fragment length polymorphism (iAFLP) (Kawamoto et al., 1999); BeadArray™ technology (Illumina, San Diego, Calif.; Oliphant et al., 2002; Ferguson et al., 2000); BeadsArray for Detection of Gene Expression (BADGE), using the commercially available Luminex100 LabMAP system and multiple color-coded microspheres (Luminex Corp., Austin, Tex.) in a rapid assay for gene expression (Yang et al., 2001); and high coverage expression profiling (HiCEP) analysis (Fukumura et al., 2003). MicroRNA Real Time PCR kits are available from Exiqon (Vedbaek, Denmark) and may be used to measure circulating miRNA in various aspects of the invention.

The miRCURY LNA™ Universal RT microRNA PCR system or other microRNA specific LNA™-based system (Exiqon; Vedbaek, Denmark) designed for sensitive and accurate detection of microRNA may also be used. Certain LNA-based systems may utilize quantitative real-time PCR using SYBR® Green. As shown in the examples below, these methods may be used to measure expression profiling of miRNAs from a smaller sample of plasma, such as less than about 1 mL or about 350 μl of plasma. Certain methods which may be used in various aspects of the invention are based on universal reverse transcription (RT) followed by real-time PCR amplification with LNA™ enhanced primers.

C. Microarrays

Other techniques for examining gene expression in a sample involve use of microarrays. Microarrays permit simultaneous analysis of a large number of gene expression products. Typically, polynucleotides of interest are plated, or arrayed, on a microchip substrate. The arrayed sequences are then hybridized with nucleic acids (e.g., DNA or RNA) from cells or tissues of interest. The source of mRNA typically is total RNA. If the source of mRNA is lung tissue, mRNA can be extracted.

In various embodiments of the microarray technique, probes to at least 10, 25, 50, 100, 200, 500, 1000, 1250, 1500, or 1600 polynucleotides are immobilized on an array substrate. The probes can include DNA, RNA, copolymer sequences of DNA and RNA, DNA and/or RNA analogues, or combinations thereof.

In some embodiments, a microarray includes a support with an ordered array of binding (e.g., hybridization) sites for each individual polynucleotide of interest. The microarrays can be addressable arrays, such as positionally addressable arrays where each probe of the array is located at a known, predetermined position on the solid support such that the identity of each probe can be determined from its position in the array.

Each probe on the microarray can be between about 10-50,000 nucleotides in length. The probes of the microarray can consist of nucleotide sequences of any length. An array can include positive control probes, such as probes known to be complementary and hybridizable to sequences in the test sample, and negative control probes such as probes known to not be complementary and hybridizable to sequences in the test sample.

Methods for attaching nucleic acids to a surface are well-known in the art. Methods for immobilizing nucleic acids on glass are described (Schena et al, 1995; DeRisi Shalon et al., 1996). Techniques are known for producing arrays with thousands of oligonucleotides at defined locations using photolithographic techniques are described by Fodor et al., 1991; Pease et al., 1994; Lockhart et al., 1996; U.S. Pat. Nos. 5,578,832; 5,556,752; and 5,510,270). Other methods for making microarrays have been described. See, e.g., Maskos and Southern, 1992. Any type of array may be used in the context of the present invention.

D. Serial Analysis of Gene Expression (SAGE)

Gene expression or miRNA expression in samples may also be determined by serial analysis of gene expression (SAGE), which is a method that allows the simultaneous and quantitative analysis of a large number of gene transcripts, without the need of providing an individual hybridization probe for each transcript (see Velculescu et al., 1995; and Velculescu et al., 1997). Briefly, a short sequence tag (about 10-14 nucleotides) is generated that contains sufficient information to uniquely identify a transcript, provided that the tag is obtained from a unique position within each transcript. Then, many transcripts are linked together to form long serial molecules, that can be sequenced, revealing the identity of the multiple tags simultaneously. The expression pattern of a population of transcripts can be quantitatively evaluated by determining the abundance of individual tags, and identifying the gene corresponding to each tag.

E. Protein Detection Methodologies

Immunohistochemical methods are also suitable for detecting the expression of the genes. Antibodies, most preferably monoclonal antibodies, specific for a gene product are used to detect expression. The antibodies can be detected by direct labeling of the antibodies themselves, for example, with radioactive labels, fluorescent labels, hapten labels such as, biotin, or an enzyme such as horse radish peroxidase or alkaline phosphatase. Alternatively, unlabeled primary antibody is used in conjunction with a labeled secondary antibody, comprising antisera, polyclonal antisera or a monoclonal antibody specific for the primary antibody. Immunohistochemistry protocols and kits are well known in the art and are commercially available.

Proteomic methods can allow examination of global changes in protein expression in a sample. Proteomic analysis may involve separation of individual proteins in a sample by 2-D gel electrophoresis (2-D PAGE), and identification of individual proteins recovered from the gel, such as by mass spectrometry or N-terminal sequencing, and analysis of the data using bioinformatics. Proteomics methods can be used alone or in combination with other methods for evaluating gene expression.

In various aspects, the expression of certain genes in a sample is detected to provide clinical information, such as information regarding prognosis. Thus, gene expression assays include measures to correct for differences in RNA variability and quality. For example, an assay typically measures and incorporates the expression of certain normalizing genes, such known housekeeping genes. Alternatively, normalization can be based on the mean or median signal (Ct) of all of the assayed genes or a large subset thereof (global normalization approach). In some embodiments, a normalized test RNA (e.g., from a patient sample) is compared to the amount found in a sample from a patient with left ventricular dysfunction. The level of expression measured in a particular test sample can be determined to fall at some percentile within a range observed in reference sets.

F. Biochips

A biochip is also provided. The biochip may comprise a solid substrate comprising an attached nucleic acid sequence that is capable of hybridizing to an miRNA sequence described herein. “Probe” as used herein may mean an oligonucleotide capable of binding to a target nucleic acid of complementary sequence through one or more types of chemical bonds, usually through complementary base pairing, usually through hydrogen bond formation. Probes may bind target sequences lacking complete complementarity with the probe sequence depending upon the stringency of the hybridization conditions. There may be any number of base pair mismatches which will interfere with hybridization between the target sequence and the single stranded nucleic acids described herein. However, if the number of mutations is so great that no hybridization can occur under even the least stringent of hybridization conditions, the sequence is not a complementary target sequence. A probe may be single stranded or partially single and partially double stranded. The strandedness of the probe is dictated by the structure, composition, and properties of the target sequence. Probes may be directly labeled or indirectly labeled such as with biotin to which a streptavidin complex may later bind. The probes may be capable of hybridizing to a target sequence under stringent hybridization conditions. The probes may be attached at spatially defined address on the substrate. More than one probe per target sequence may be used, with either overlapping probes or probes to different sections of a particular target sequence. The probes may be capable of hybridizing to target sequences associated with a single disorder. The probes may be attached to the biochip in a wide variety of ways, as will be appreciated by those in the art. The probes may either be synthesized first, with subsequent attachment to the biochip, or may be directly synthesized on the biochip.

The solid substrate may be a material that may be modified to contain discrete individual sites appropriate for the attachment or association of the probes and is amenable to at least one detection method. Representative examples of substrates include glass and modified or functionalized glass, plastics (including acrylics, polystyrene and copolymers of styrene and other materials, polypropylene, polyethylene, polybutylene, polyurethanes, TeflonJ, etc.), polysaccharides, nylon or nitrocellulose, resins, silica or silica-based materials including silicon and modified silicon, carbon, metals, inorganic glasses and plastics. The substrates may allow optical detection without appreciably fluorescing.

The substrate may be planar, although other configurations of substrates may be used as well. For example, probes may be placed on the inside surface of a tube, for flow-through sample analysis to minimize sample volume. Similarly, the substrate may be flexible, such as a flexible foam, including closed cell foams made of particular plastics.

The biochip and the probe may be derivatized with chemical functional groups for subsequent attachment of the two. For example, the biochip may be derivatized with a chemical functional group including, but not limited to, amino groups, carboxyl groups, oxo groups or thiol groups. Using these functional groups, the probes may be attached using functional groups on the probes either directly or indirectly using a linkers. The probes may be attached to the solid support by either the 5′ terminus, 3′ terminus, or via an internal nucleotide.

The probe may also be attached to the solid support non-covalently. For example, biotinylated oligonucleotides can be made, which may bind to surfaces covalently coated with streptavidin, resulting in attachment. Alternatively, probes may be synthesized on the surface using techniques such as photopolymerization and photolithography

### IV. KITS

The technology herein includes kits for evaluating miRNA or gene expression in samples. A “kit” refers to a combination of physical elements. For example, a kit may include, for example, one or more components such as probes, including without limitation specific primers, antibodies, a protein-capture agent, a reagent, an instruction sheet, and other elements useful to practice the technology described herein. These physical elements can be arranged in any way suitable for carrying out the invention.

Kits for analyzing RNA expression may include, for example, a set of oligonucleotide probes for detecting expression of miR-21, miR-210, miR-155, and/or miR-196a. The probes can be provided on a solid support, as in an array (e.g., a microarray), or in separate containers. The kits can include a set of oligonucleotide primers useful for amplifying a set of genes described herein, such as to perform PCR analysis. Kits can include further buffers, enzymes, labeling compounds, and the like. Any of the compositions described herein may be comprised in a kit. The kit may further include water and hybridization buffer to facilitate hybridization of the two strands of the miRNAs.

A kit for analyzing protein expression can include specific binding agents, such as immunological reagents (e.g., an antibody) for detecting protein expression of a gene of interest. The components of the kits may be packaged either in aqueous media or in lyophilized form. The container means of the kits will generally include at least one vial, test tube, flask, bottle, syringe or other container means, into which a component may be placed, and preferably, suitably aliquoted. Where there is more than one component in the kit, the kit also will generally contain a second, third or other additional container into which the additional components may be separately placed. However, various combinations of components may be comprised in a single vial. The kits of the present invention also will typically include a means for containing the nucleic acids, and any other reagent containers in close confinement for commercial sale. Such containers may include injection or blow-molded plastic containers into which the desired vials are retained.

When the components of the kit are provided in one and/or more liquid solutions, the liquid solution is an aqueous solution, such as a sterile aqueous solution.

However, the components of the kit may be provided as dried powder(s). When reagents and/or components are provided as a dry powder, the powder can be reconstituted by the addition of a suitable solvent. It is envisioned that the solvent may also be provided in another container means.

The container means will generally include at least one vial, test tube, flask, bottle, syringe and/or other container means, into which the nucleic acid formulations are placed, preferably, suitably allocated. The kits may also comprise a second container means for containing a sterile, pharmaceutically acceptable buffer and/or other diluent.

The kits of the present invention will also typically include a means for containing the vials in close confinement for commercial sale.

Such kits may also include components that preserve or maintain nucleotides which can bind an miRNA or that protect against miRNA degradation. Such components may be RNAse-free or protect against RNAses. Such kits generally will comprise, in suitable means, distinct containers for each individual reagent or solution.

A kit will also include instructions for employing the kit components as well the use of any other reagent not included in the kit. Instructions may include variations that can be implemented.

It is contemplated that such reagents are embodiments of kits of the invention. Such kits, however, are not limited to the particular items identified above and may include any reagent used for the manipulation or characterization of miRNA.

### V. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Materials and Methods

The three different study population screened in our studies consisted of patients with pathologically confirmed primary pancreatic ductal adenocarcinoma, chronic pancreatitis and controls recruited at The University of Texas M. D. Anderson Cancer Center, TexGen consortium and at the Mayo Clinic, Jacksonville. Controls were healthy spouses, friends, or non-blood relatives of patients with various non-gastrointestinal and non-smoking related cancers. Controls were frequency-matched to cases by age at enrollment (±5 years), sex, and race. All study subjects gave written informed consent for the interviews and the collection of blood sample in accordance with the protocols approved by the Institutional Review Board of M. D. Anderson Cancer Center and the Mayo Clinic. Total of 49 cancer and 36 control samples of Heparin treated plasma collected between 2002 and 2008; 29 cancer, 9 chronic pancreatitis and 22 control samples of EDTA treated plasma collected between 2002 and 2004; 50 cancer, 19 chronic pancreatitis and 19 control pancreatic juice samples collected between 2007 and 2009 were analyzed in this study. The patient population characteristics with respect to age, race, sex, stage of disease and survival durations of those profiled for miRNAs in plasma and pancreatic juice are described in Table 1 and 2.

TABLE 2
Patient Characteristics of pancreatic juice samples

| | Normal controls | Pancreatitis (CP) | PDAC |
|---|---|---|---|
| Characteristic | (n = 19) | (n = 19) | (n = 50) |
| Age (medium) | 65 | 65 | 67.5 |
| Sex | | | |
| Male | 5 | 11 | 30 |
| Female | 14 | 8 | 20 |
| Race | | | |
| White | 16 | 17 | 48 |
| Black | 2 | 1 | 2 |
| Hispanic | 1 | 0 | 0 |
| Asian | 0 | 1 | 0 |
| Smoking history ^{a} | | | |
| Current | 2 | 7 | 9 |
| Former | 6 | 6 | 15 |
| Never | 11 | 6 | 25 |
| History of alcohol use ^{a} | | | |
| Current | 9 | 9 | 24 |
| Former | 5 | 5 | 8 |
| Never | 5 | 5 | 17 |
| Diabetes mellitus ^{a} | | | |
| Yes | 3 | 5 | 10 |
| No | 16 | 14 | 39 |

^{a} Smoking history, history of alcohol and Diabetes use data were not available for 1 patient.

Collection of Heparin and EDTA Treated Blood Plasma

Blood was collected from patients and controls in Sodium Heparin tubes (BD Vacutainer, Franklin Lakes, N.J.) and processed within 2 h of collection by centrifugation at 1,300×g at 4° C. for 10 min. Plasma was transferred to a fresh tube and stored at −80° C.

Collection of Pancreatic Juice

Pancreatic juice was collected from the ampulla of patients and controls following i.v. administration of synthetic human secretin with 7Fr catheter for 10 min. Collected juice was transferred into fresh tubes as 2 ml aliquots with 1 tablet of protease inhibitor cocktail (Roche), snap frozen and stored at −80° C.

RNA Isolation

Total RNA containing small RNA was isolated from 300 μl-1.5 ml of plasma using Trizol LS reagent (Invitrogen Life Technologies, CA) according to the manufacturer's protocol with the following modifications. The plasma and the pancreatic juice samples were mixed with Trizol LS reagent (1:3 ratio for plasma and 1:2 ratio for pancreatic juice), and after phase separation by centrifugation, the upper aqueous phase was carefully transferred to a fresh tube. The aqueous phase was then extracted twice with phenol/chloroform and added with 1.5 vol of ethanol before being applied directly to mirVana miRNA column (Ambion, Inc, Austin, Tex.) according to the manufacturer's instructions. The bound RNA was cleaned with the buffers provided by the manufacturer to remove impurities and eluted in a final volume of 100 μl. To remove heparin associated contaminants from the heparin treated plasma, 300 μl of 7.5 M LiCl was added to the RNA solution, incubated overnight at −20° C. and then centrifuged at 12,000×g for 30 min at 4° C. The pellet was washed two times by centrifugation with 70% ethanol. The RNA pellet was dried for 10 min at room temperature and dissolved in 30 μl of DEPC-treated water for miRNA assay. The concentration of all RNA samples were quantified using NanoDrop 1000 (Nanodrop, Wilmington, USA).

Plasma RNA Pretreatment

20 ng of RNA was pretreated with 1 unit of DNase (Invitrogen, CA) for 1 h at 25° C. in 1 μl of 10×DNase I Reaction Buffer (200 mM Tris-HCl pH 8.4, 20 mM MgCl2, 500 mM KCl) in presence of 0.38 μl of RNAse inhibitor to remove any contaminating DNA. For the Heparin treated plasma 2 units of Heparinase I (Sigma Chemical Company) was added to remove the Heparin. After the enzyme digestions, 0.5 μl of 25 mM EDTA solution was added to the total reaction volume of 10 μl and incubated at 65° C. for 10 min.

MicroRNA Real Time PCR

Taqman MicroRNA Assays were used to do expression profiling of the plasma miRNAs of interest. All reagents, primers and probes were obtained from Applied Biosystems (Applied Biosystems, Foster City, Calif.). 10 ng of DNAse and Heparinase treated plasma RNA for each sample was used for the individual assays in 15 μl reactions containing RT mixture and Taqman primer mix. The mix was incubated at 16° C. for 30 min, 42° C. for 60 min, and 85° C. for 5 min. miRNA expression levels were quantified using the ABI prim 7900 HT Sequence detection system (Applied Biosystems). For the purpose, 15 μl reverse transcription (RT) reaction was diluted with 30 μl of water and 11.25 μl of the diluted RT product was mixed with 12.5 μl of 2×Taqman PCR mixture, 1.25 μl Taqman primer and probe mixture in a final volume of 25 ul. Real time PCR was performed in triplicate, including no-template controls. Relative expression of the mature miRNAs was calculated utilizing the comparative CT (2-ΔΔCT) method (Schmittgen and Livak, 2008) with miRNA-16 as the endogenous control to normalize the data (Wong et al., 2008). The cycle threshold (CT) is defined as the number of cycles required for the FAM signal to cross the threshold in real time PCR. ΔCT was calculated by subtracting the CT values of miR-16 from the CT values of the miRNA of interest. ΔΔCT was then calculated by subtracting mean ΔCT of the control samples from ACT of tested samples. Fold change of miRNA was calculated by the equation 2^{-ΔΔCT}.

Pancreatic Juice Micro RNA Real Time PCR

Taqman MicroRNA Assays were used to do expression profiling of the Pancreatic juice miRNAs of interest. All reagents, primers and probes were obtained from Applied Biosystems (Applied Biosystems, Foster City, Calif.). 15 ng of DNAse treated pancreatic juice RNA for each sample was used for the individual assays in 15 μl reactions containing RT mixture and Taqman primer mix. The mix was incubated at 16° C. for 30 min, 42° C. for 60 min, and 85° C. for 5 min. miRNA expression levels were quantified using the ABI prim 7900 HT Sequence detection system (Applied Biosystems). For the purpose, 15 μl reverse transcription (RT) reaction was diluted with 30 μl of water and 3.15 μl of the diluted RT product was mixed with 3.5 μl of 2×Taqman PCR mixture, 0.35 μl Taqman primer and probe mixture in a final volume of 7 ul. Real time PCR was performed in triplicate, including no-template controls. Relative expression of the mature miRNAs was calculated utilizing the comparative C_{T} (2^{-ΔΔCT}) method with RNU6B as the endogenous control to normalize the data for the pancreatic juice samples. The cycle threshold (CT) is defined as the number of cycles required for the FAM signal to cross the threshold in real time PCR. ΔCT was calculated by subtracting the CT values of RNU6B from the CT values of the miRNA of interest. ΔΔCT was then calculated by subtracting mean ΔCT of the control samples from ΔCT of tested samples. Fold change of miRNA was calculated by the equation 2^{-ΔΔCT}.

Statistical Analysis

Student's t-test was used to evaluate expression differences of miRNAs between cases and controls. Fisher's exact test and Pearson's Chi-Square Test were used to determine if there was significant association between the relative plasma levels of the four miRNAs. All tests of statistical significance were two-sided. P-values of less than 0.05 were considered statistically significant. Receiver operating characteristic curves (ROC) were constructed and the area under the curve (AUC) was calculated to evaluate the specificity and sensitivity of predicting cases and controls by each individual miRNA and by the combination of the four miRNAs. Since this study population was small, the cases and controls were not split into training and test sets. We, however, used a leave-one-out scheme to cross-validate the ROC analysis. In the leave-one-out analysis a subset of all but one observation is used to build a model, and then the model is used to predict the left-out recorded observation. When this process is repeated for each observation, a prediction is obtained for every record in the data set using a model that was blind to the predicted observation. These predictions generate a table of statistics that is used for cross-validating the ROC analysis. All statistical analyses were performed using the Stata 8.0 software (Stata Corporation, College Station, Tex.).

### Example 2

### MicroRNAs in Plasma of Pancreatic Ductal Adenocarcinoma Patients are Blood Based Biomarkers of Disease

A modified RNA isolation protocol for real time RT-PCR assay of plasma derived miRNAs from blood collected in heparin tubes was performed as described above. This protocol yielded 100 ng-500 ng of total RNA from 1.5 ml plasma samples. The isolated RNA samples could quantify relative miRNA levels in a reproducible manner as evident from the results of at least two repeated experiments of every sample run in either duplicate or triplicate in each instance. The results of these independent experiments did not show significant differences (t-test, P=0.41). Furthermore, Pearson's correlation coefficient revealed significant positive correlation between the relative miRNA levels quantified in independent experiments (r=0.705, P=0.0002). The modifications introduced in the published methods to eliminate heparin and other contaminants including DNA were critical for successful real time RT-PCR reactions for plasma miRNAs. To improve the reproducibility of real time PCR results, some samples were processed through multiple cycles of purification steps, which affected the final yield of RNA isolated in each case. Since miR-16 has been reported to be one of the most stably expressed miRNAs across 40 normal human tissue types (Liang et al., 2007) that is detectable at modest levels in normal human plasma (Mitchell et al., 2008) and given that expression of miR-16 has also been used as the calibrator for assaying relative miRNA expression in human tissues, it was used as the endogenous control in these experiments. It was observed that the cycle threshold (CT) values for miR-16 did not vary significantly (P>0.05) in the different reaction batches for control and cancer plasma samples thus validating miR-16 as a reliable endogenous control. Due to this observation, the inventors routinely ran the real time RT-PCR reactions for miR-16 in triplicate first to check the quality of each plasma RNA sample and reproducibility of their assay performance before analyzing them for additional miRNAs of interest. In this study, besides assaying for miR-16 as the endogenous normalization control we selected a panel of four miRNAs implicated in pancreatic cancer, miR-21, miR-210, miR-155 and miR-196a, to interrogate their plasma levels in 49 pancreatic cancer patients and 36 normal healthy individuals. However, due to varying yield of the isolated RNA in each case, a total of 28 cancer and 19 control samples could be finally analyzed for all the four miRNAs.

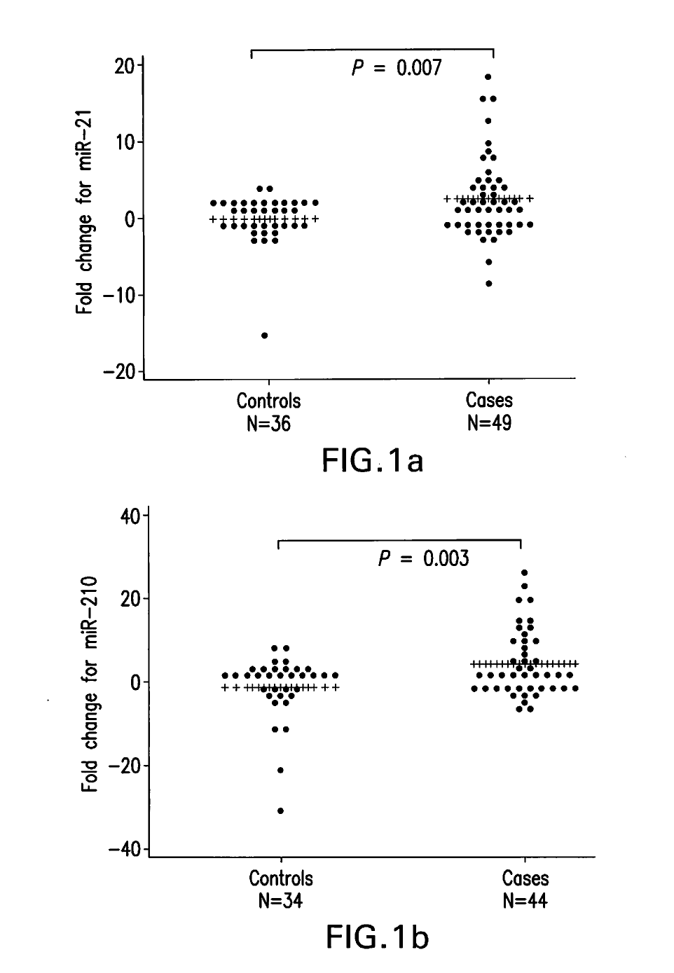

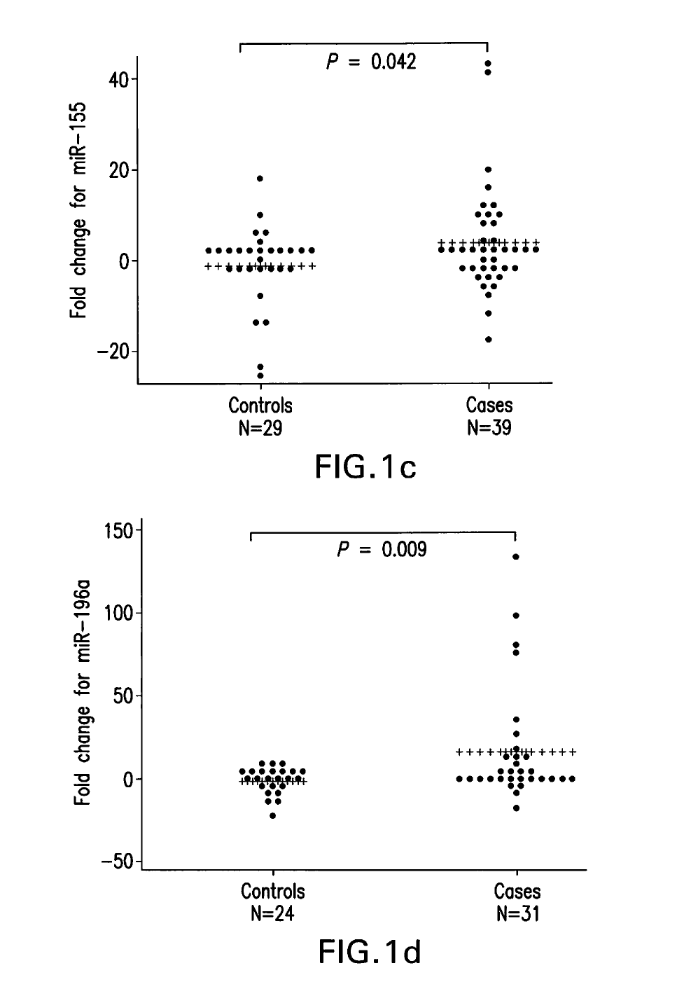

The relative levels of miR-21, miR-210, miR-155 and miR-196a normalized to the level of the miR-16 endogenous control were elevated overall in the plasma of pancreatic adenocarcinoma patients (FIGS. 1A-D). The mean fold changes (2-ΔΔCt) in relative levels and P values reflected segregation between normal healthy controls and PDAC samples. Differences in the mean fold change for each miRNA was, however, also a function of the relative abundance of the respective miRNAs in plasma. While the distribution of miR-21, miR-210 and miR-155 levels were spread over a broader range in the healthy controls, those of miR-196a were significantly narrower with its abundance being distinctly less in all the control samples. For miR-21, thirty one of the forty nine cancer samples revealed about 2-20 fold elevation in plasma levels while among the controls, nineteen of the thirty six displayed only 2-4 fold increase. Regarding miR-210, twenty eight of the forty four cancer cases had plasma levels elevated by about 2-28 fold but the increase was limited to 2-8 fold in twenty one of the thirty four control samples. A 2-40 fold increase of miR-155 was detected in twenty three of the thirty nine cancer cases as opposed to 2-18 fold increase in sixteen of the twenty nine controls. With the relative abundance of miR-196a being low in most of the control plasma, the cancer samples revealed elevation in plasma levels ranging from about 5-140 fold in fifteen of the thirty one cases compared with only about 5-10 fold elevation seen in ten of the twenty four control samples. With the rest of the samples showing either no change or negative fold change values, the overall mean fold increase for each of the four miRNAs in the plasma of cancer samples compared with the controls were significant with the P values of 0.007 for miR21, 0.003 for miR-210, 0.042 for miR-155 and 0.009 for miR-196a (Table 3).

TABLE 3
Mean Fold Change of Plasma miRNA Levels
in PDAC and Control Samples

| | PDAC | Control | |
|---|---|---|---|
| | Mean Fold | Mean Fold | |
| miRNA | change ± SEM | change ± SEM | p-value |
| miR-21 | 2.42 ± 0.76 | −0.13 ± 0.54 | 0.007 |
| miR-210 | 4.22 ± 1.19 | −1.56 ± 1.33 | 0.003 |
| miR-155 | 3.74 ± 1.81 | −1.31 ± 1.63 | 0.042 |
| miR-196a | 16.05 ± 6.11 | −1.56 ± 1.63 | 0.009 |

Examination of individual mean fold increases in the cancer samples indicated that a relatively limited number of outliers contributed greatly to the overall highly significant differences observed between the cancer and the control samples. However, no significant differences in the plasma levels of the four miRNAs, both individually and in combination, (P-value >0.20) were observed for the cancer samples at different stages of the disease. These observations suggested that the overall mean fold increase in plasma miRNA levels were detected even in patients with localized disease and was, therefore, not a marker of patients with only late stage metastatic cancer.

Interestingly, with a five-fold or more increase as the cutoff for individual samples, the data revealed a significant association between the elevated plasma levels of miR-155 and miR-210 in cancer patients (p-value=0.004). For miR-155, eleven of the thirty nine (28%) and for miR-210, fourteen of the forty four (32%) cancer samples showed increase in plasma levels spanning this range. Of these, eight cancer samples revealed identical increases in relative plasma levels for miR-155 (73%) and miR-210 (57%).

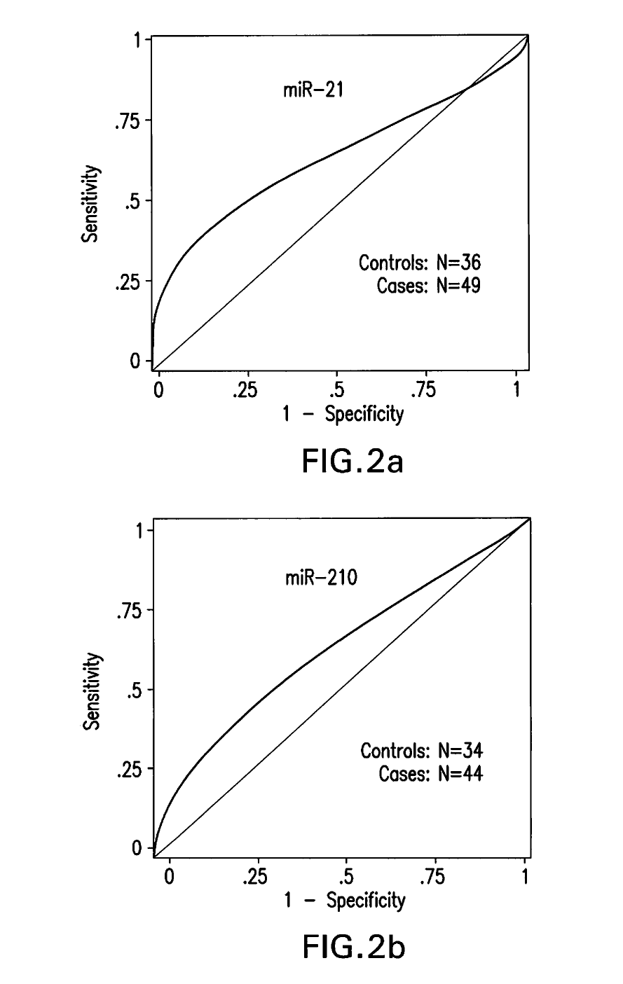

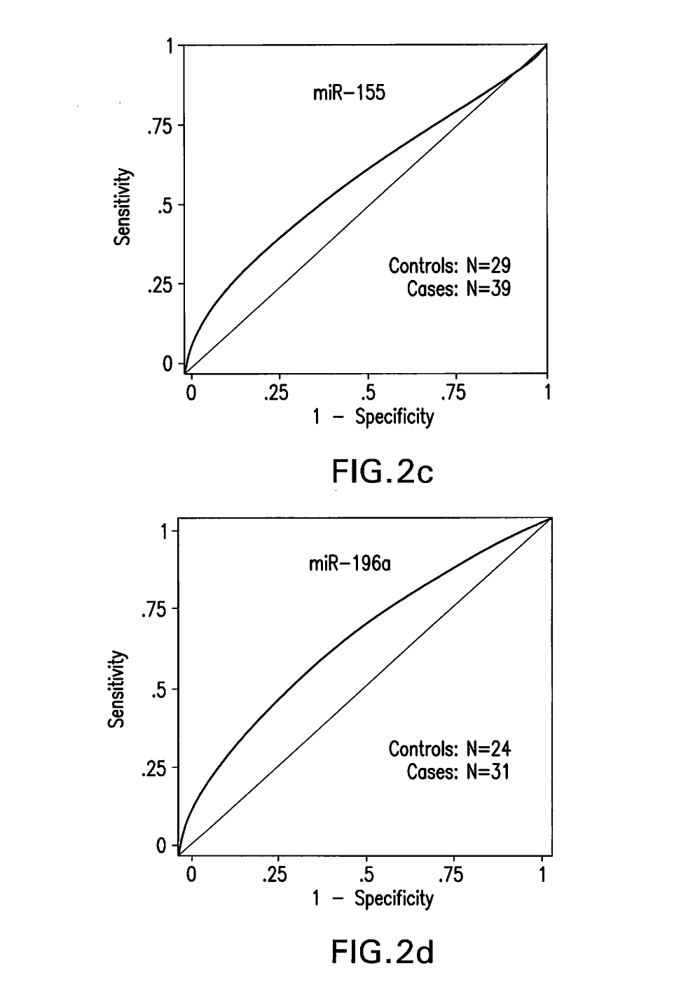

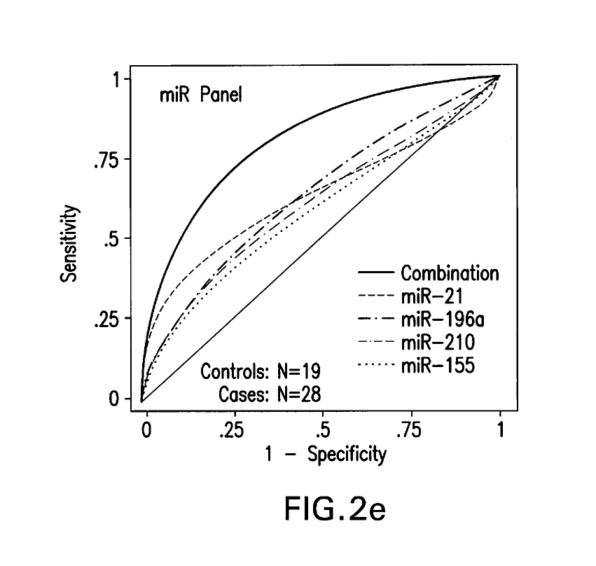

In order to determine if the relative fold changes in the four plasma miRNAs could significantly differentiate between pancreatic cancer patients and healthy controls, receiver operating characteristic (ROC) curves were constructed (FIGS. 2A-D). The area under the ROC curve (AUC) for miR-21 was 0.63 (95% confidence interval [CI]: 0.51-0.75); for miR-210 was 0.62 (95% CI: 0.49-0.74); for miR-155 was 0.60 (95% CI: 0.46-0.74); and for miR-196a was 0.66 (95% CI: 0.51-0.80). As mentioned above, due to varying yields of the isolated RNAs in each case, a total of 28 cancer and 19 control samples could be finally analyzed for all the four miRNAs. We separately compared the AUC for each miRNA and the combination of the four miRNAs in this sample set also. Results revealed that there was a highly significant difference in the AUC values obtained for the four individual miRNAs and the panel of four in combination (p=0.0008) in this set of 28 cancer and 19 control samples. The area under the ROC curve (AUC) for miR-21 was 0.62 (95% confidence interval [CI]: 0.45-0.77); for miR-210 was 0.65 (95% CI: 0.49-0.80); for miR-155 was 0.67 (95% CI: 0.51-0.82); and for miR-196a was 0.69 (95% CI: 0.53-0.84). Remarkably, the AUC for the combination of these four miRNAs was 0.82 (95% CI: 0.70-0.94). Thus the AUC increased from 0.62-0.69 range for each individual miRNA to 0.82 for the four miRNAs combined (FIG. 2E). The ROC curves also helped determine the sensitivities and specificities for the plasma miRNAs at various cut-off values. Using the optimal cut-point, the sensitivity and specificity were 46% and 89% for miR-21; 42% and 73% for miR-210; 53% and 78% for Mir155; and 43% and 84% for Mir196a respectively. On the other hand, the sensitivity and specificity for the four miRNAs combined were 64% and 89%. Finally, the composite panel of the four miRNAs in plasma revealed a sensitivity of 46% given a specificity of 100% and a specificity of 37% given a sensitivity of 100% in this study.

Since the sample size in the experiments was small, we applied leave-one-out scheme to further validate our ROC results. The estimate of the AUC, as obtained by leave-one-out cross-validation, was 0.78 (95% CI 0.64-0.91) for the four miRNAs combined, which still showed a good discriminating power. Using the optimal cut-point, the sensitivity and specificity for the four miRNAs combined were 64% and 89% after cross-validation, which is same as that obtained before cross-validation. Similarly, the composite panel of the four miRNAs in plasma revealed a sensitivity of 46% given a specificity of 100% and a specificity of 32% given a sensitivity of 100% after cross-validation. The results, therefore, document that the combined analysis of this four miRNAs in a panel had a reasonable power to differentiate pancreatic cancer patients from healthy controls.

### Example 3

### MicroRNAs in Pancreatic Juice of Chronic Pancreatitis and Pancreatic Ductal Adenocarcinoma Patients are Biomarkers of Disease

The RT-PCR assay of pancreatic juice derived miRNAs was performed as described in the protocol. There was significant correlation between the relative miRNA levels in independent experiments. In this study, we profiled the same panel of four miRNAs described above along with RNU6B as the endogenous normalization control in 50 pancreatic cancer patients (PC), 19 chronic pancreatitis patients (CP) and 19 normal controls.

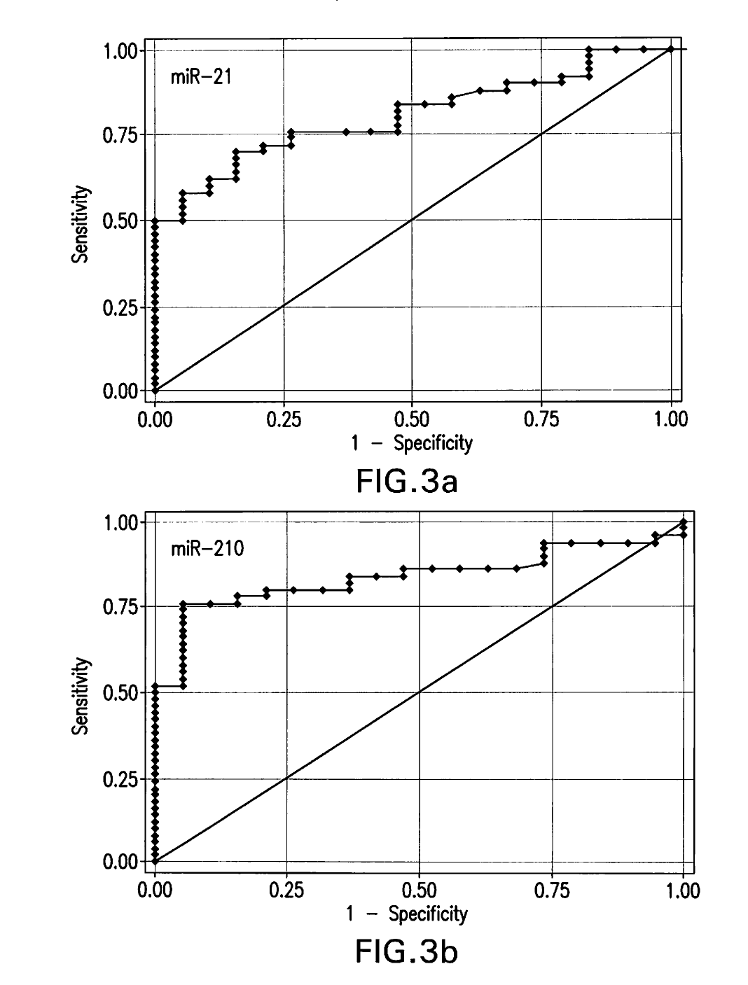

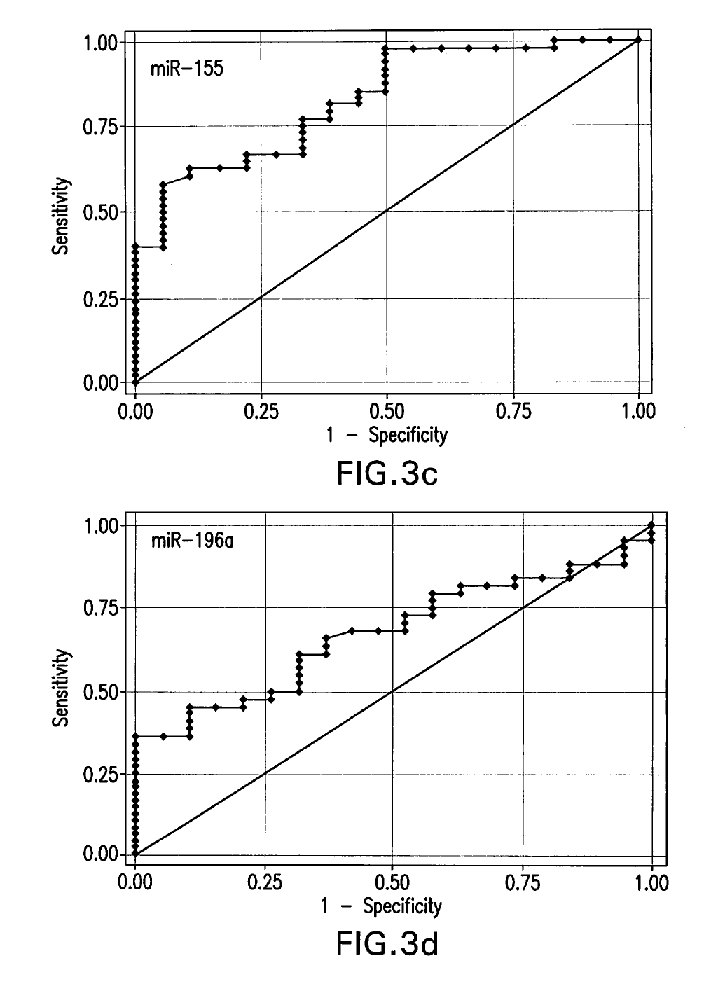

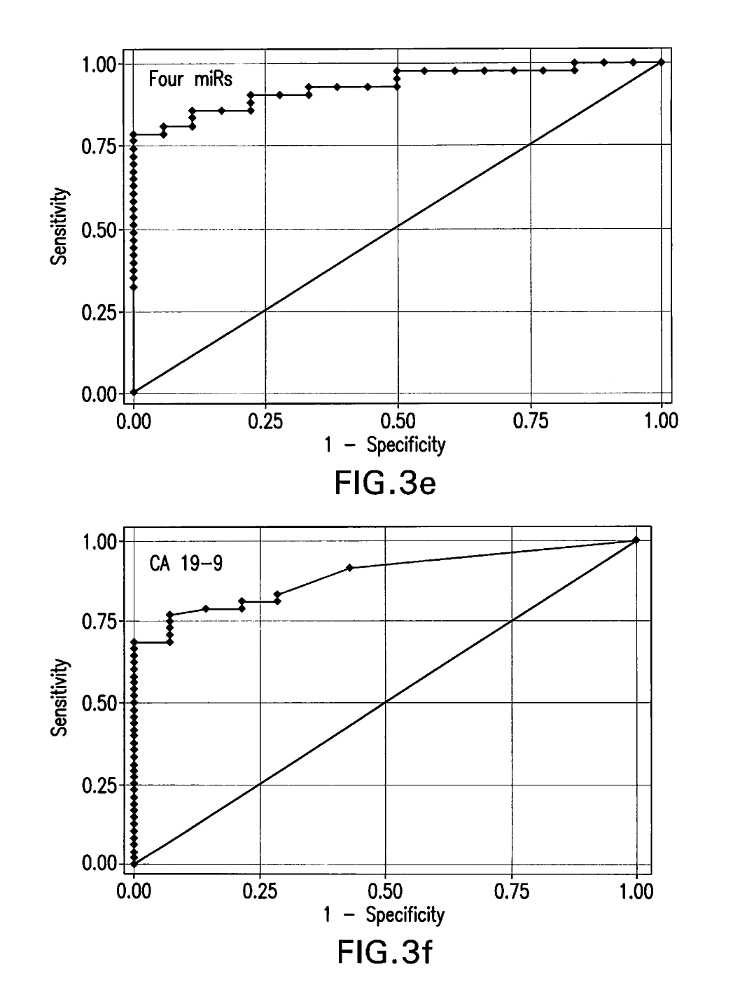

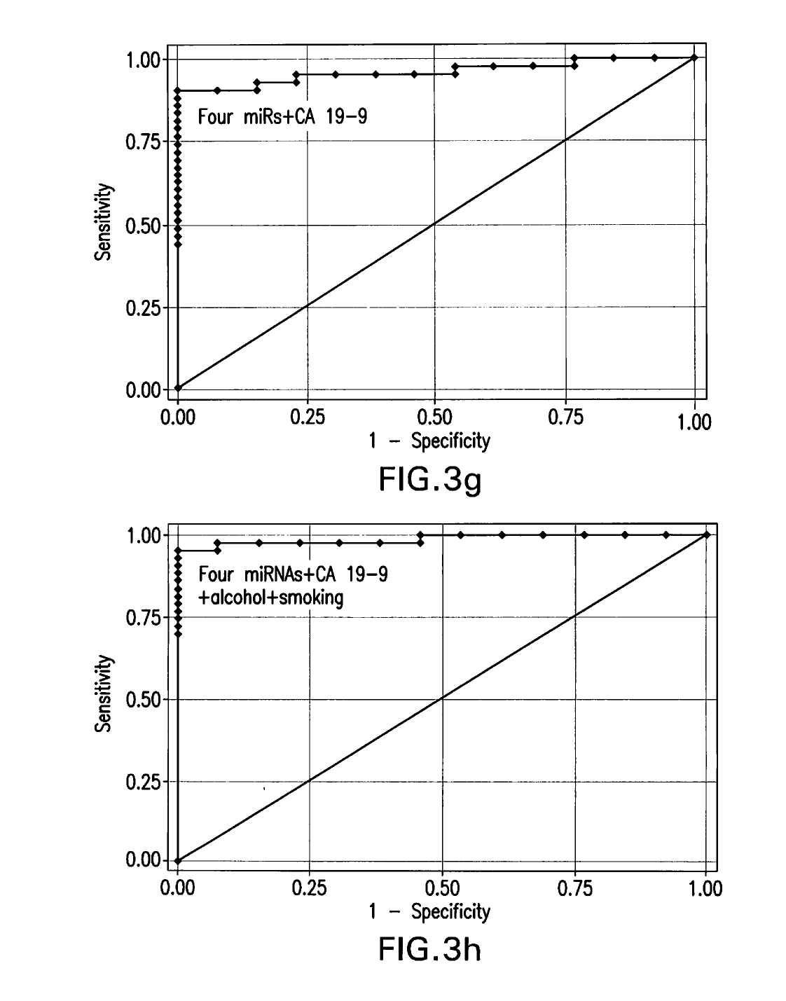

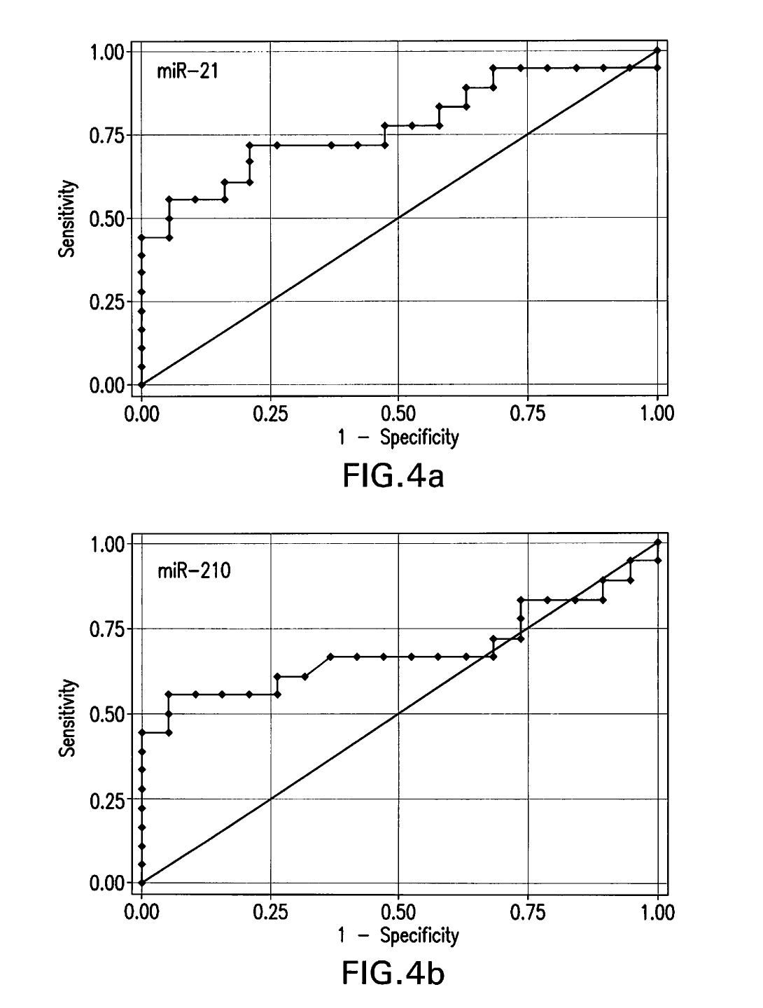

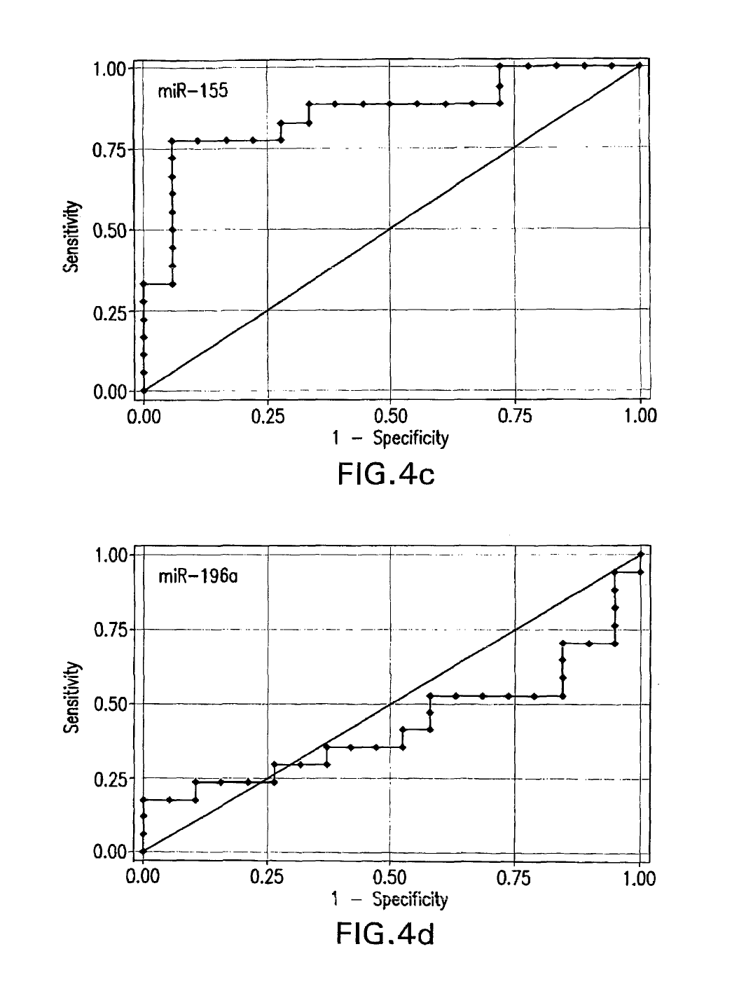

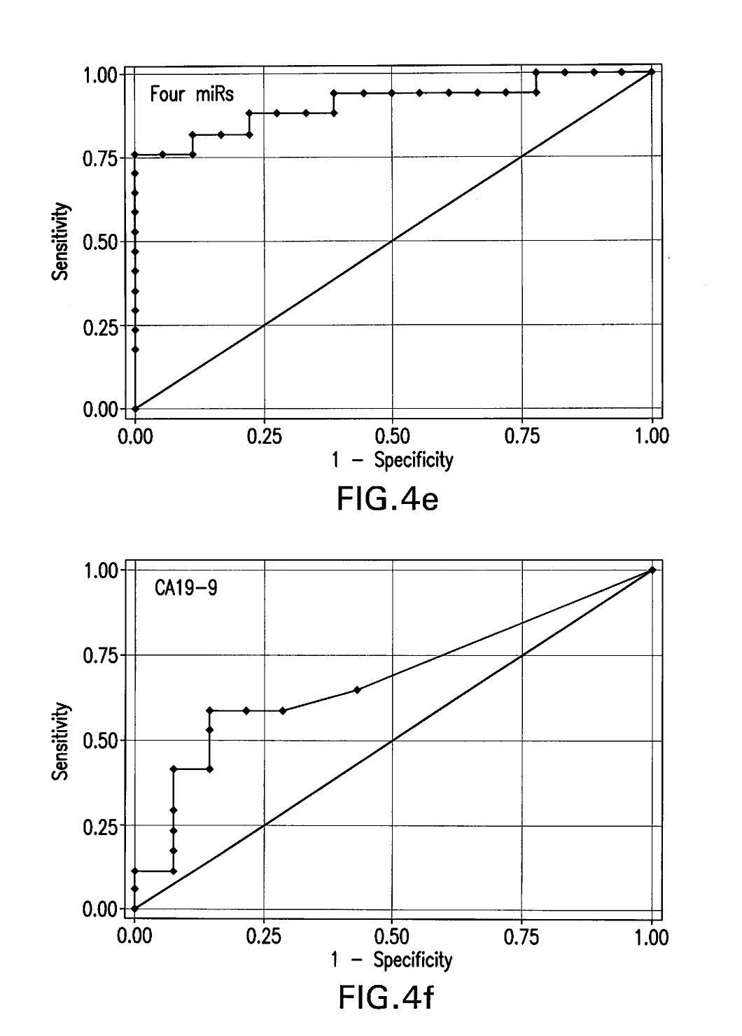

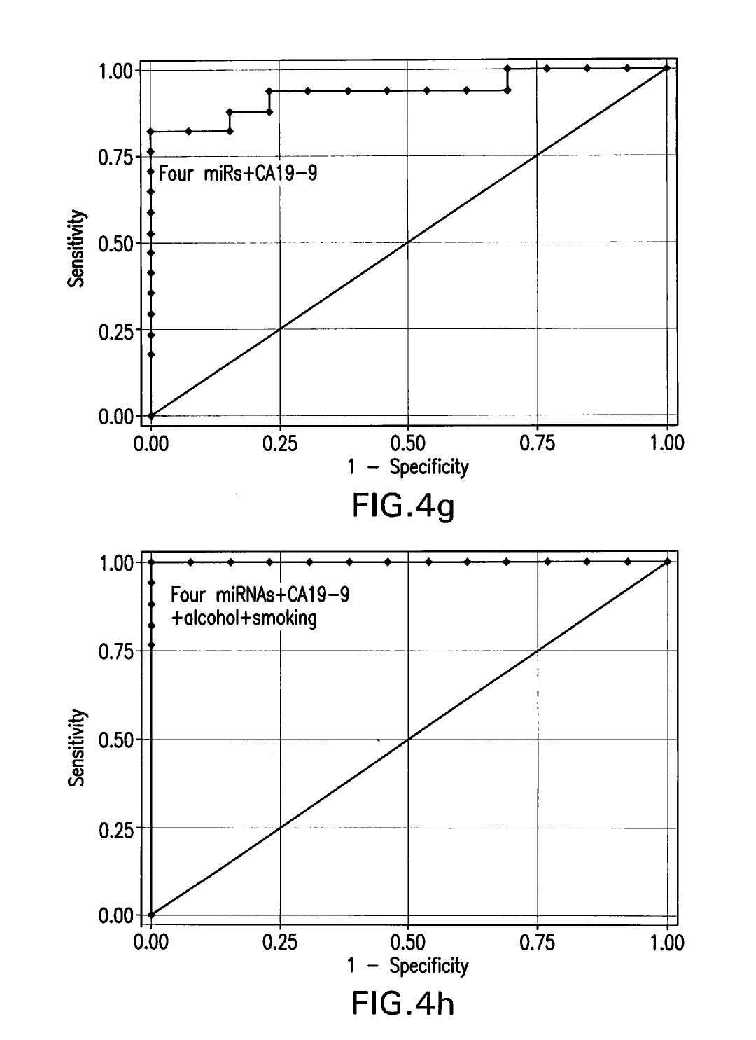

The relative levels of miR-21, miR-210, miR-155 and miR-196a normalized to the levels RNU6B endogenous control were elevated overall in the pancreatic adenocarcinoma patients (FIG. 3) but in case of chronic pancreatitis only miR-21, miR-210 and miR-155 were elevated at significant levels compared to the normal control subjects (FIG. 4). The overall mean fold increase for each of the four miRNAs in the pancreatic juice of cancer samples compared with the controls were significant with the P values of 0.04 for miR-21, 0.005 for miR-210, <0.001 for miR-155 and 0.010 for miR-196a (Table-4).

TABLE 4
Mean Fold Change of Pancreatic Juice miRNA
Levels in PDAC and Control Samples.

| | PDAC (n = 50) | Control (n = 19) | |
|---|---|---|---|
| miRNA | Mean Fold change ± SE | Mean Fold change ± SE | p-value |
| miR-21 | 62.30 ± 250.27 | −1.04 ± 6.49 | 0.040 |
| miR-210 | 587.91 ± 1551.67 | 1.97 ± 9.25 | 0.005 |
| miR-155 | 39.89 ± 61.72 | 0.39 ± 8.26 | <0.001 |
| miR-196a | 16.62 ± 45.22 | 0.16 ± 2.78 | 0.010 |

In case of chronic pancreatitis patients the over all mean fold increase was significant for miR-21 (P=0.020), miR-210 (P=0.025) and miR-155 (P=0.001) while the mean fold change was not significant (P=0.151) for miR-196a (Table-5).

TABLE 5
Mean Fold Change of Pancreatic Juice miRNA Levels
in Pancreatitis (CP) and Control Samples.

| | CP (n = 19) | Control (n = 19) | |
|---|---|---|---|
| miRNA | Mean Fold change ± SE | Mean Fold change ± SE | p-value |
| miR-21 | 11.30 ± 23.63 | −1.04 ± 6.49 | 0.020 |
| miR-210 | 574.02 ± 1191.59 | 1.97 ± 9.25 | 0.025 |
| miR-155 | 31.92 ± 39.04 | 0.39 ± 8.26 | 0.001 |
| miR-196a | 4.05 ± 15.26 | 0.16 ± 2.78 | 0.151 |

The ROC curves helped determine the sensitivity and specificity for the pancreatic juice miRNA at various cut-off values. Using the optimal cut-point, the sensitivity and specificity between normals and PC for the 4 miRNA panel was 86% and 89% while for 4 miRNA panel+19-9+alcohol+smoking was 98% and 92%.

Between normal and CP, the sensitivity and specificity for the 4 miRNA panel was 82% and 88%, while both sensitivity and specificity for 4 miRNA panel+19-9+alcohol and smoking was 100%.

In order to determine if the relative fold change in the four plasma miRNAs could significantly differentiate between pancreatic cancer (PC) patients and controls as well as between chronic pancreatitis (CP) and controls, receiver operating characteristic (ROC) curves were constructed (FIGS. 3, 4). The area under the ROC curve (AUC) for miR-21 was 0.81 for PC and 0.77 for CP; for miR-210 was 0.84 for PC and 0.68 for CP; for miR-155 was 0.83 for PC and 0.86 for CP and for miR-196a was 0.68 for PC and 0.43 for CP relative to the controls respectively. Due to varying yields of the RNAs from individual samples, a total of 50 PC, 19 CP and 19 controls could be analyzed for all the four miRNAs. Results further revealed that there was a highly significant difference in the AUC values for the four individual miRNAs and the panel of four in combination. Remarkably, the AUC for the combination of these four miRNAs was 0.93 for the PC and 0.91 for the CP samples relative to the controls.

Additionally, we used Kruskal-Wallis test to evaluate differences in mIRNAs among the groups with respect to their alcohol consumption and smoking history. Then pair-wise comparison was conducted when a significant value was found in each of the groups. These investigations demonstrated that the change in miRNA levels in pancreatic juice segregate significantly with PC and CP groups identified with alcohol consumption and smoking history and thus can identify individuals at risk of developing PC and CP if they have a history of alcohol consumption and smoking.

As observed in the results:

i) PC patients with history of alcohol consumption reveal significantly higher miR-21 and miR-210 values than in normals (P<0.001), while no significant difference was observed for those without history of alcohol consumption.

ii) CP patients with history of alcohol consumption have significantly higher miR-21 (P<0.001), miR-210 (P<0.005) and miR-155 (<0.001) than normal controls, while among those without alcohol history there no significant differences were observed in the levels of these miRNAs between patients and controls.

iii) CP patients with smoking history show significantly higher miR-21 values than normals (P<0.005) while among those with no smoking history there no significant difference in the level of miR-21 was detected.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
U.S. Pat. No. 5,034,506
U.S. Pat. No. 5,235,033
U.S. Pat. No. 5,510,270
U.S. Pat. No. 5,556,752
U.S. Pat. No. 5,578,832
U.S. Pat. No. 6,268,490
U.S. Pat. No. 6,770,748
U.S. Patent Publn. 2002/0115080
U.S. Patent Publn. 2005/0107325
U.S. Patent Publn. 2005/0182005
Adsay et al., Semin. Radiat. Oncol., 15:254-64, 2005.
Asangani et al., Oncogene, 27:2128-36, 2008.
Ausubel et al., Current Protocols in Molecular Biology, Greene Publ. Assoc. Inc. & John Wiley & Sons, Inc., MA, 1997.
Bloomston et al., JAMA, 297:1901-8, 2007.
Chan et al., Cancer Res., 65:6029-33, 2005.
Chang and Mendell, Annu. Rev. Genomics Hum. Genet., 8:215-39, 2007.
Chen et al., Int. J. Cancer, 120:1511-7, 2007.
Croce and Calin, Cell, 122:6-7, 2005.
Crosby et al., Cancer Res., 69:1221-9, 2009.
De Andres et al., BioTechniques 18:42044, 1995.
DeRisi Shalon et al., Genome Res., 6:639-645, 1996.
DiGiuseppe et al., Am. J. Clin. Pathol., 101:684-688, 1994.
Ding and Cantor, Proc. Natl. Acad. Sci. USA, 100:3059-3064, 2003.
Eloubeidi et al., Cancer, 99:285-92, 2003.
Faca et al., PLoS Med., 5:953-967, 2008.
Ferguson et al., Proc. Natl. Acad. Sci. USA, 97(11):6049-6054, 2000.
Fodor et al., Science, 251:767-773, 1991.
Frankel et al., Bio. Chem., 283:1026-33, 2008.
Fukumura et al., Nucl. Acids. Res., 31(16):e94, 2003.
Gironella et al., Proc. Natl. Acad. Sci. USA, 104:16170-5, 2007.
Godfrey et al., J. Molec. Diagn., 2: 84-91, 2000.
Goggins, Semin. Oncol., 34:303-10, 2007.
Habbe et al., Cancer Biol. Ther., 8:340-6, 2009.
Hanash et al., Nature, 452:571-579, 2008.
Hod, Biotechniques, 13:852 854, 1992.
Hruban et al., Am. J. Surg. Pathol., 28:977-87, 2004.
Hruban et al., Gastroenterol. Clin North Am., 36:831-49, 2007.
Hwang and Mendell, Br. J. Cancer, 94:776-80, 2006.
Jhala et al., Am. J. Clin. Pathol., 126:572-9, 2006.
Jhala et al., Cancer, 102:239-46, 2004.
Kawamoto et al., Genome Res., 12:1305-1312, 1999.
Krutzfeldt et al., Nature, 438(7068):685-689, 2005.
Lee et al., Int. J. Cancer, 120:1046-54, 2007.
Liang and Pardee, Science, 257:967-971, 1992.
Liang et al., BMC Genomics, 8:166-185, 2007.
Lockhart et al., Nat. Biotechnol., 14(13):1675-1680, 1996.
Lüttges et al., Cancer, 85:1703-1710, 1999.
Lynam-Lennon et al., Biol. Rev. Camb. Philos. Soc., 84:55-71, 2009.
Matsuo et al., International Journal of Cancer, 125: 1027-1037, 2009.
Maitra and Hruban, Annu. Rev. Pathol., 3:157-88, 2008.
Maitra et al., Adv. Anat. Pathol., 12:81-91, 2005.
Maskos and Southern, Nuc. Acids. Res., 20:1679-1, 684, 1992.
Mitchell et al., Proc. Natl. Acad. Sci. USA, 105:10513-8, 2008.
Noh et al. Clin Gastroenterol Hepatol. 4(6):782-9, 2006.
Oliphant et al., In: Discovery of Markers for Disease (Supplement to Biotechniques), June 2002.
Parker and Barnes, Methods Mol. Biol., 106:247-283, 1999.
Pease et al., Proc. Natl. Acad. Sci. USA, 91:5022-5026, 1994.
Pungpapong et al., Pancreatology. 7(5-6):491-6, 2007.
Roldo et al., J. Clin. Oncol., 24:4677-84, 2006.
Rupp and Locker, Lab Invest., 56:A67, 1987.
Schena et al, Science, 270:467-470, 1995.
Schmittgen and Livak, Nat. Protoc., 3:1101-8, 2008.
Soutschek et al., Nature, 432:173-178, 2004.
Specht et. al., Am. J. Pathol., 158: 419-29, 2001.
Szafranska et al., Clin. Chem., 54:1716-24, 2008.
Szafranska et al., Oncogene, 26:4442-52, 2007.
Van Gulik et al., Gastrointest Endosc., 46:417-23, 1997.
Velculescu et al., Cell, 88:243-51, 1997.
Velculescu et al., Science, 270:484-487, 1995.
Vilmann and Saftoiu, J. Gastroenterol. Hepatol., 21:1646-55, 2006.
Volinia et al., Proc. Natl. Acad. Sci. USA, 103:2257-61, 2006.
Weis et al., Trends in Genetics, 8:263 264, 1992.
Wilentz et al., Cancer Res., 60:2002-2006, 2000.
Wong et al., Clin. Cancer Res., 14:2588-92, 2008.
Yang et al., Cancer Res., 68:425-33, 2008.
Yang et al., Genome Res., 11:1888-1898, 2001.

1-31. (canceled)

32. A method of detecting for the presence or an increased risk of a pancreatic cancer in a subject, comprising:
(a) obtaining a biological sample that comprises miRNA sequences of the subject; and
(b) measuring the level of miR-21, miR-210, miR-155, or miR-196a in the biological sample, wherein an increased level of miR-21, miR-210, miR-155, or miR-196a relative to a normal control indicates an increased risk of or the presence of a pancreatic cancer in the subject, and wherein the biological sample comprises a blood sample, a plasma sample, a serum sample, or a pancreatic juice sample.

33. The method of claim 32, wherein said measuring comprises measuring the level of said miR-21, miR-210, miR-155, or miR-196a using RT-PCR, a biochip, quantitative PCR, serial analysis of gene expression (SAGE), or a microarray.

34. The method of claim 32, wherein said measuring comprises measuring the level of at least two of said miR-21, miR-210, miR-155, or miR-196a.

35. The method of claim 34, wherein said measuring comprises measuring the level of at least three of said miR-21, miR-210, miR-155, or miR-196a.

36. The method of claim 32, wherein said measuring comprises measuring the level of each of said miR-21, miR-210, miR-155 and miR-196a.

37. The method of claim 32, wherein the subject is a human.

38. The method of claim 37, wherein the human has pancreatic cancer.

39. The method of claim 37, wherein the human has pancreatitis or chronic pancreatitis.

40. The method of claim 37, further comprising a method of discriminating between a pancreatic cancer and pancreatitis in the patient, wherein an increase in the level of miR-196a relative to a normal control indicates that the subject indicates an increased risk of or the presence of a pancreatic cancer in the subject; wherein no increase in the level of miR-196a, and an increase in at least one of miR-21, miR-210, or miR-155 indicates that the subject has pancreatitis or chronic pancreatitis.

41. A method for monitoring the progression of a pancreatic cancer in a subject comprising:
(a) obtaining a first biological sample from the subject;
(b) subsequently obtaining a second biological sample from the subject; and
(c) measuring miR-21, miR-210, miR-155, or miR-196a levels in said first and second biological samples;
wherein an increase in the expression of miR-21, miR-210, miR-155, or miR-196a in the second sample relative to the first sample indicates an adverse disease progression of the pancreatic cancer in the patient, and wherein a decrease in the expression of miR-21, miR-210, miR-155, or miR-196a in the second sample relative to the first sample indicates disease regression of the pancreatic cancer in the patient, wherein the biological sample comprises a blood sample, a serum sample, or a pancreatic juice sample.

42. The method of claim 41, wherein said measuring comprises using RT-PCR, a biochip, quantitative PCR, serial analysis of gene expression (SAGE), or a microarray.

43. The method of claim 41, wherein the method further comprises measuring at least two of miR-21, miR-210, miR-155, or miR-196a in said first and second biological samples.

44. The method of claim 43, wherein the method further comprises measuring at least three of miR-21, miR-210, miR-155, or miR-196a in said first and second biological samples.

45. The method of claim 43, wherein said measuring comprises measuring expression of said miR-21, miR-210, miR-155, and miR-196a in said first and second biological samples.

46. The method of claim 41, wherein the patient is administered an anti-cancer therapy.

47. The method of claim 46, wherein the anti-cancer therapy is selected from the group consisting of a surgery, a chemotherapy, a radiation therapy, a gene therapy, a protein therapy, or an immunotherapy.

48. A kit comprising a sealed container comprising primers or probes designed to detect specific for transcription or reverse transcription of at least two of miR-21, miR-210, miR-155, or miR-196a.

49. The kit of claim 48, comprising primers or probes designed to detect at least three of miR-21, miR-210, miR-155, or miR-196a.

50. The kit of claim 49, comprising primers or probes designed to detect each of miR-21, miR-210, miR-155, and miR-196a.

51. The kit of claim 48, wherein said kit further comprises one or more reagents for RT-PCR or reverse transcription RT-PCR.

52. A biochip comprising an isolated nucleic acid comprising at least two of miR-21, miR-210, miR-155, or miR-196a, or a complement thereof.

53. The biochip of claim 52, comprising at least three of miR-21, miR-210, miR-155, or miR-196a, or its complement.

54. The biochip of claim 52, comprising each of miR-21, miR-210, miR-155, or miR-196a, or its complement.

55. A method for discriminating or distinguishing between a pancreatic cancer and pancreatitis in a human subject comprising:
(a) obtaining a biological sample from the subject;
(b) measuring miR-196a levels in the biological sample, and measuring at least one of miR-21, miR-210, miR-155 in the biological sample;
wherein an increase in the expression of miR-196a in the sample relative to a normal control indicates the presence of or an increased risk of a pancreatic cancer in the subject; and
wherein the lack of an increase in the level of miR-196a relative to a normal control, and an increase in one or more of miR-21, miR-210, or miR-155 relative to a normal control, indicates that the subject has the presence of or an increased of pancreatitis;
wherein the biological sample comprises a blood sample, a serum sample, or a pancreatic juice sample.

56. The method of claim 55, wherein said measuring comprises using RT-PCR, a biochip, quantitative PCR, serial analysis of gene expression (SAGE), or a microarray.

57. The method of claim 55, wherein the method comprises measuring at least two of miR-21, miR-210, and miR-155 in the biological sample.

58. The method of claim 57, wherein the method comprises measuring at least three of miR-21, miR-210, and miR-155 in the biological sample.

59. The method of claim 57, wherein the method comprises measuring all of miR-21, miR-210, and miR-155 in the biological sample.

60. The method of claim 55, wherein the subject has pancreatic cancer.

61. The method of claim 60, wherein the pancreatic cancer comprises an exocrine tumor.

62. The method of claim 61, wherein the pancreatic cancer comprises an endocrine tumor.

63. The method of claim 55, wherein the subject has pancreatitis.

64. The method of claim 63, wherein the pancreatitis is chronic pancreatitis.

65. The method of claim 55, wherein the subject is administered an anti-cancer therapy.

66. The method of claim 65, wherein the anti-cancer therapy is selected from the group consisting of a surgery, a chemotherapy, a radiation therapy, a gene therapy, a protein therapy, or an immunotherapy.


SEQUENCE LISTING (13 sequences)

SEQ ID NO: 1 - RNA, 22 nt, Homo sapiens
uagcuuaucagacugauguuga

SEQ ID NO: 2 - RNA, 22 nt, Homo sapiens
cugugcgugugacagcggcuga

SEQ ID NO: 3 - RNA, 23 nt, Homo sapiens
uuaaugcuaaucgugauaggggu

SEQ ID NO: 4 - RNA, 22 nt, Homo sapiens
uagguaguuucauguuguuggg

SEQ ID NO: 5 - RNA, 22 nt, Homo sapiens
uaauacugccugguaaugauga

SEQ ID NO: 6 - RNA, 22 nt, Homo sapiens
ugauauguuugauauauuaggu

SEQ ID NO: 7 - RNA, 22 nt, Homo sapiens
caaagaauucuccuuuugggcu

SEQ ID NO: 8 - RNA, 23 nt, Homo sapiens
agcuacauugucugcuggguuuc

SEQ ID NO: 9 - RNA, 21 nt, Homo sapiens
agcuacaucuggcuacugggu

SEQ ID NO: 10 - RNA, 22 nt, Homo sapiens
uagcagcacaucaugguuuaca

SEQ ID NO: 11 - RNA, 22 nt, Homo sapiens
uucaacggguauuuauugagca

SEQ ID NO: 12 - RNA, 22 nt, Homo sapiens
ugucaguuugucaaauacccca

SEQ ID NO: 13 - RNA, 22 nt, Homo sapiens
gugaaauguuuaggaccacuag